SEQUENCE LISTING (3 sequences)

SEQ ID NO: 1 - protein, 427 aa, Eubacterium rectale
MDTSNLMEQILSSDNLNRAYLQVVRNKGAEGVDGMKYTELKEHLAKNGETIKGQLRTRKYKPQPARRVEIPKPDGGVRNLGVPTVTDRFIQQAIAQVLTPIYEEQFHDHSYGFRPNRCAQQAILTALNIMNDGNDWIVDIDLEKFFDTVNHDKLMTLIGRTIKDGDVISIVRKYLVSGIMIDDEYEDSIVGTPQGGNLSPLLANIMLNELDKEMEKRGLNFVRYADDCIIMVGSEMSANRVMRNISRFIEEKLGLKVNMTKSKVDRPSGLKYLGFGFYFDPRAHQFKAKPHAKSVAKFKKRMKELTCRSWGVSNSYKVEKLNQLIRGWINYFKIGSMKTLCKELDSRIRYRLRMCIWKQWKTPQNQEKNLVKLGIDRNTARRVAYTGKRIAYVCNKGAVNVAISNKRLASFGLISMLDYYIEKCVTC

SEQ ID NO: 2 - DNA, 42 bp, synthetic construct
  source 1..42: mol_type = other DNA
ccttctccttctcctcctttctcctttttttttttttttttt

SEQ ID NO: 3 - DNA, 28 bp, synthetic construct
  source 1..28: mol_type = other DNA
ccctctctctctctttcctctctctttt

(19) United States
(12) Patent Application Publication
(10) Pub. No.: US 2026/0085309 A1
(43) Pub. Date: Mar. 26, 2026

(54) Compositions for cDNA Synthesis and Transcriptome Profiling and Methods of Use Thereof

(71) Applicant: YALE UNIVERSITY, New Haven, CT (US)

(72) Inventors: Anna Marie Pyle, New Haven, CT (US); Li-Tao Guo, New Haven, CT (US)

(21) Appl. No.: 19/111,674

(22) PCT Filed: Sep. 14, 2023
(86) PCT No.: PCT/US2023/074203

§ 371 Date: Mar. 13, 2025

Related U.S. Application Data
(60) Provisional application No. 63/375,592, filed on Sep. 14, 2022.

(51) Int. Cl. C12N 15/10; C12N 9/12; C12P 19/34; C12Q 1/6848

(52) U.S. Cl. CPC C12N 15/1096; C12N 9/1276; C12P 19/34; C12Q 1/6848; C12Y 207/07049

(57) ABSTRACT

The present invention provides optimized template switching oligonucleotides, methods, and kits for performing reverse transcription. The optimized template switching oligonucleotides include modifications of 5′ and 3′ ends to prevent the formation of concatemers and to enhance the specificity of reverse transcription.

### CROSS REFERENCE TO RELATED APPLICATIONS

This application claims priority to U.S. Provisional Application No. 63/375,592, filed Sep. 14, 2022 which is hereby incorporated by reference herein in its entirety.

### STATEMENT REGARDING FEDERALLY SPONSORED RESEARCH OR DEVELOPMENT

This invention was made with government support under HG011868 awarded by National Institutes of Health. The government has certain rights in the invention.

### BACKGROUND OF THE INVENTION

RNA sequencing has become the standard method for transcriptome profiling. It provides a far more precise measurement of levels and diversity of transcripts and their isoforms than other methods. When the terminal sequences of transcripts are unknown, or when a sequencing library is prepared from a very limited amount of RNA material, e.g., total RNA from a single cell, robust full-length cDNA synthesis and adapter addition are crucial to produce a representative, non-biased source of nucleic acid material from the transcriptome under investigation. In this case, the reverse transcriptase (RT) template switching reaction has been exploited to simultaneously enrich full-length cDNA and add two different adapters to both 5′- and 3′-end of cDNA molecules during first strand synthesis, which overcomes the many shortcomings of ligation-based approaches. Single-cell applications utilizing template switching are dependent on the efficiency and specificity of the reverse transcription and the template switching reaction. Due to the ultra-low amount of RNA input, the products of non-specific reverse transcription from template switching oligo, and template switching oligo concatemerization dominate the sequencing libraries, thereby limiting application of this approach.

Thus, there is a need in the art for improved compositions to promote specificity and yield of template-switching based RNA-seq library preparation. The present invention addresses this unmet need.

### SUMMARY OF THE INVENTION

The present disclosure features template switching oligonucleotides (TSO), as well as compositions comprising the same, and related methods of use thereof. In one embodiment, the template switching oligonucleotide (TSO) comprises a DNA nucleotide sequence, an RNA nucleotide sequence, a modified nucleotide sequence or a hybrid DNA-RNA sequence. In an embodiment, the TSO can anneal by base-pairing to non-templated nucleotides, for example, non-templated nucleotides added to the 5′-end of a target nucleic acid molecule. In an embodiment, the non-templated nucleotides are added to the target nucleic acid by a reverse transcriptase. In an embodiment, the TSO further comprises at least one of a 3′ end modification and a 5′ end modification.

In an embodiment, the TSO comprises a 3′ end modification. In an embodiment, the 3′ end modification may either be a modification to remove the 3′ hydroxyl group or a modification to block the 3′ hydroxyl group. In one embodiment, the TSO comprises a 3′ end modification selected from the group consisting of 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1).

In an embodiment, the TSO comprises a 5′ end modification. In one embodiment, the 5′ end is modified with a chemical group to reduce or substantially block concatemerization. In one embodiment, the 5′ end is modified with a chemical group selected from the group consisting of trityl, trebbler, a dendrimer, biotin, a fluorescent dye, ROX NHS ester, a (CH2)n (n≥1) long spacer, palmitate phosphoramidite, 3-cyanovinylcarbazole phosphoramidite, cholesteryl, and psoralen.

In one embodiment, the 5′ end comprises at least 10, 9, 8, 7, 6, 5, 4, 3, or 2 consecutive abasic sites. In one embodiment, the 5′ end comprises at least 10 consecutive abasic sites. In one embodiment, the 5′ end comprises at least 8 consecutive abasic sites. In one embodiment, the 5′ end comprises at least 6 consecutive abasic sites. In one embodiment, the 5′ end comprises at least 5 consecutive abasic sites. In one embodiment, the 5′ end comprises at least 4 consecutive abasic sites. In one embodiment, the 5′ end comprises at least 3 consecutive abasic sites. In one embodiment, the 5′ end comprises at least 2 consecutive abasic sites.

In one embodiment, the 5′ end comprises at least one non-natural nucleotide or nucleotide analog. In one embodiment, the nucleotide at the 5′ end is a non-natural nucleotide or a nucleotide analog. In one embodiment, the TSO comprises at least one isodeoxycytosine (iso-dC), isodeoxyguanosine (iso-dG) or a combination of iso-dC and iso-dG at the 5′ end.

In one embodiment, the TSO comprises at least one 3′ end modification and at least one 5′ end modification.

In one embodiment, the disclosure relates to a reverse transcription (RT) primer comprising a DNA nucleotide sequence, an RNA nucleotide sequence, a modified nucleotide sequence or a hybrid DNA-RNA sequence, wherein the RT primer comprises a 5′ end modification. In one embodiment, the 5′ end is modified with a chemical group to block concatemerization. In one embodiment, the 5′ end is modified with a chemical group selected from the group consisting of 5′ trebbler, and 5′ trityl. In one embodiment, the 5′ end comprises at least 3 consecutive abasic sites. In one embodiment, the nucleotide at the 5′ end is a non-natural nucleotide or a nucleotide analog. In one embodiment, the RT primer comprises at least one isodeoxycytosine (iso-dC), isodeoxyguanosine (iso-dG) or a combination of iso-dC and iso-dG at the 5′ end.

In one embodiment, the disclosure relates to a method of generating a cDNA molecule from an RNA template, the method comprising contacting an RNA template with a TSO comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification, a reverse transcription (RT) primer and a reverse transcriptase.

In one embodiment, the RT primer comprises a DNA nucleotide sequence, an RNA nucleotide sequence, a modified nucleotide sequence or a hybrid DNA-RNA sequence, wherein the RT primer comprises a 5′ end modification. In one embodiment, the 5′ end is modified with trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, or psoralen.

In one embodiment, the reverse transcriptase is MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, or TGIRT™ or a variant thereof.

In one embodiment, the method is included in an RT-PCR, qRT-PCR, capillary electrophoresis (CE) for RNA-structure mapping, transcriptome profiling, in-cell sequencing, next-generation RNA sequencing (RNA-seq), nanopore sequencing, PacBio sequencing, zero-mode waveguide sequencing, cDNA library synthesis, or cDNA synthesis assay, or any combination thereof.

In one embodiment, the invention relates to a reverse transcription assay for of generating a cDNA molecule from an RNA template, the method comprising contacting an RNA template with a TSO comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification, a reverse transcription (RT) primer and a reverse transcriptase.

In one embodiment, the RT primer is selected from the group consisting of a DNA primer, an RNA primer, and a primer comprising at least one modified oligonucleotide.

In one embodiment, the reverse transcriptase is MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, or TGIRT™ or a variant thereof.

In one embodiment, the reverse transcription is performed in a buffer comprising PEG8000. In one embodiment, the reverse transcription is performed in a buffer comprising LiCl.

In one embodiment, the invention relates to a kit for performing an assay for generating a cDNA molecule from an RNA template, the method comprising contacting an RNA template with a TSO comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification, a reverse transcription (RT) primer and a reverse transcriptase.

In one embodiment, the RT primer is a DNA primer, an RNA primer, or a primer comprising at least one modified oligonucleotide.

In one embodiment, the reverse transcriptase is MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, and TGIRT™ or a variant thereof. In one embodiment, the kit comprises a buffer comprising PEG8000. In one embodiment, the kit comprises a buffer comprising LiCl.

In one embodiment, the invention relates to a template switching oligonucleotide (TSO) comprising:
(i) a DNA nucleotide sequence, an RNA nucleotide sequence, or a hybrid DNA-RNA sequence; and
(ii) at least one of a 3′ end modification and a 5′ end modification.

In one embodiment, the TSO comprises a 3′ end modification. In one embodiment, the TSO comprises a 5′ end modification. In one embodiment, the TSO comprises both a 3′ end modification and a 5′ end modification.

In one embodiment, the 3′ end modification is a nucleotide sugar modification or a nucleobase modification.

In one embodiment, the 3′ end modification is a modification to remove the 3′ hydroxyl group or a modification to block the 3′ hydroxyl group.

In one embodiment, the 3′ end modification is 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), or 3′ (CH2CH2O)n (n≥1).

In one embodiment, the 5′ end modification is a nucleotide sugar modification or a nucleobase modification.

In one embodiment, the 5′ end modification comprises trityl, trebbler, a dendrimer, biotin, a fluorescent dye, ROX NHS ester, (CH2)n (n≥1) long spacer, palmitate phosphoramidite, 3-cyanovinylcarbazole phosphoramidite, cholesteryl, or psoralen.

In one embodiment, the 3′ end modification is a modification to remove the 3′ hydroxyl group or a modification to block the 3′ hydroxyl group.

In one embodiment, the 5′ end comprises at least 2, 3, 4, 5, 6, 7, 8, 9, or 10 consecutive abasic sites. In one embodiment, the 5′ end comprises at least 5 consecutive abasic sites. In one embodiment, the 5′ end comprises at least 3 consecutive abasic sites.

In one embodiment, the 5′ end comprises at least one non-natural nucleotide or nucleotide analog.

In one embodiment, the TSO comprises at least one isodeoxycytosine (iso-dC), isodeoxyguanosine (iso-dG) or a combination of iso-dC and iso-dG at the 5′ end.

In one embodiment, the invention relates to a method of reducing the concatemerization of a template switching oligonucleotide (TSO), the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

In one embodiment, the concatemerization is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

In one embodiment, the 3′ end modification is 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), or 3′ (CH2CH2O)n (n≥1).

In one embodiment, the TSO comprises a 5′ end modification of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, or psoralen.

In one embodiment, the 5′ end of the TSO comprises at least 3 consecutive abasic sites.

In one embodiment, the TSO comprises at least one isodeoxycytosine (iso-dC), isodeoxyguanosine (iso-dG) or a combination of iso-dC and iso-dG at the 5′ end.

In one embodiment, the TSO comprises SEQ ID NO: 3.

In one embodiment, the RT primer comprises SEQ ID NO: 2.

In one embodiment, the invention relates to a method of reducing the non-specific reverse transcription from a template switching oligonucleotide (TSO), the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

In one embodiment, the concatemerization is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

In one embodiment, the 3′ end modification is 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), or 3′ (CH2CH2O)n (n≥1).

In one embodiment, the TSO comprises a 5′ end modification of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, or psoralen.

In one embodiment, the 5′ end of the TSO comprises at least 3 consecutive abasic sites.

In one embodiment, the TSO comprises at least one isodeoxycytosine (iso-dC), isodeoxyguanosine (iso-dG) or a combination of iso-dC and iso-dG at the 5′ end.

In one embodiment, the TSO comprises SEQ ID NO: 3.

In one embodiment, the RT primer comprises SEQ ID NO: 2.

In one embodiment, the invention relates to a method of increasing yield of target polynucleotide sequences in a RNA-seq library, the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

In one embodiment, the concatemerization is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

In one embodiment, the 3′ end modification is 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), or 3′ (CH2CH2O)n (n≥1).

In one embodiment, the TSO comprises a 5′ end modification of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, or psoralen.

In one embodiment, the 5′ end of the TSO comprises at least 3 consecutive abasic sites.

In one embodiment, the TSO comprises at least one isodeoxycytosine (iso-dC), isodeoxyguanosine (iso-dG) or a combination of iso-dC and iso-dG at the 5′ end.

In one embodiment, the TSO comprises SEQ ID NO: 3.

In one embodiment, the RT primer comprises SEQ ID NO: 2.

In one embodiment, the invention relates to a method of increasing the specificity of an RNA-seq library, the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

In one embodiment, the concatemerization is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

In one embodiment, the 3′ end modification is 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), or 3′ (CH2CH2O)n (n≥1).

In one embodiment, the TSO comprises a 5′ end modification of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, or psoralen.

In one embodiment, the 5′ end of the TSO comprises at least 3 consecutive abasic sites.

In one embodiment, the TSO comprises at least one isodeoxycytosine (iso-dC), isodeoxyguanosine (iso-dG) or a combination of iso-dC and iso-dG at the 5′ end.

In one embodiment, the TSO comprises SEQ ID NO: 3.

In one embodiment, the RT primer comprises SEQ ID NO: 2.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 provides a schematic of the design of the template switching oligo (TSO). The 5′ and 3′ modifications are designed to prevent unwanted reactions during RNA sequencing library preparation.

FIG. 2 provides an overview of a template switching reaction without TSO 5′ modification in which TSO concatemers form.

FIG. 3 provides an overview of a template switching reaction with TSO 5′ modification in which TSO concatemerization is reduced. The 5′ modification (*) prevents the second TSO from annealing to the ‘AAA’ overhang.

FIG. 4 depicts various modifications that have been demonstrated to be effective in reducing TSO concatemerization. These include five consecutive abasic sites (5′-5abasic), three consecutive abasic sites (5′-3abasic), a trebbler group (5′-trebbler) and a trityl group (5′-trityl).

FIG. 5A and FIG. 5B depict data demonstrating the quantification of TSO concatemerization products using the TSOs with 5′ modifications. FIG. 5A depicts an electrophoretic analysis of template switching products using the TSOs without (lane 1) or with 5′ modifications (lane 2-5). The number of concatenated TSOs for each template switching product was denoted in the figure. FIG. 5B depicts the quantification of the efficiency of multiple template switching reactions (≥2, orange) using the TSOs without (bar 1) or with 5′ modifications (bar 2-5).

FIG. 6 depicts a schematic of TSO 3′ modification which prevents the TSO from serving as a primer. When an RNA-seq library is prepared using an ultra-low RNA input (e.g., total RNA from a single cell), the cDNA products derived from TSO priming will dominate the RNA-seq library. 3′ modifications (e.g., a dideoxy nucleotide) that block the 3′-OH of TSO can effectively block the unwanted reaction.

FIG. 7A through FIG. 7C depict the nucleotide sequence specificity of non-templated nucleotide addition (NTA) by different reverse transcriptases. FIG. 7A depicts the nucleotide sequence specificity of Marathon RT. FIG. 7B depicts the nucleotide sequence specificity of MMLV RT. FIG. 7C depicts the nucleotide sequence specificity of TGIRT. The sequence of the TSO can be selected based on the standard NTA by the reverse transcriptase. MarathonRT specifically adds a triple-adenosine overhang to the 3′-end of cDNA. It needs a TSO with three uridines (RNA) or three thymidines (DNA) at the 3′-end for efficient template switching. Moloney Murine Leukemia Virus (MMLV) RTs add a triple-cytidine overhang to the 3′-end of cDNA. It needs a TSO with three guanosines at the 3′-end for efficient template switching. TGIRT™ most efficiently adds a single nucleotide overhang (a mixture of A, G, C and T) to the 3′-end of cDNA. A TSO with any nucleotide at the 3′-end can mediate template switching.

### DETAILED DESCRIPTION

The present invention provides compositions and methods to control the non-specific reverse transcription and template switching products while maintaining the high efficiency of full-length cDNA synthesis. Key components used for reverse transcription and template switching reaction have been optimized to increase the yield of reverse transcription reactions. Specifically, a highly processive RT encoded by Eubacterium rectale group II intron is used in place of a retroviral RT to increase full-length cDNA yield and reduce bias during reverse transcription and template switching; the reaction buffer components are optimized to increase the template switching efficiency for higher yield of full-length cDNA; the nucleotide sequences for the RT primer and template switching oligo are optimized and chemical modifications were made at the 3′-end of the template switching oligo to reduce non-specific reverse transcription; and chemical modifications were made to and a non-standard nucleotide included at the 5′-end of the template switching oligo to prevent concatemerization during template switching.

In one aspect, the present invention provides a composition comprising an optimized template switching oligonucleotide (TSO) comprising at least one 3′ end modification, at least one 5′ end modification, or a combination of at least one 3′ end modification and at least one 5′ end modification.

In one embodiment, the TSO comprises a 3′ end modification. Exemplary 3′ end modifications include, but are not limited to, 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1). In one embodiment, the TSO comprises at least one chemical group that blocks the 3′ hydroxyl group. In one embodiment, the TSO comprises at least one modification that removes the 3′ hydroxyl group.

In one embodiment, the TSO comprises at least one 5′ end modification. In some embodiments the TSO comprises a combination of 5′ end modifications.

In one embodiment, the TSO comprises at least one non-standard nucleotide. In some embodiments, at least one non-standard nucleotide is at the 5′-end of the TSO. In one embodiment, the non-standard nucleotide is an isodeoxycytosine.

In one embodiment, the TSO comprises at least one chemical group that blocks the 5′ end. In some embodiments, the chemical group comprises a bulky adduct that prevents binding of the reverse transcriptase and additional TSO molecules to the single stranded overhang on the template molecule. Exemplary chemical groups that can be added to the TSO to block the 5′ end to prevent concatemerization include, but are not limited to, trityl, dendrimers (for example, trebbler), biotin, fluorescent dyes, ROX NHS ester, (CH2)n (n≥1) long spacer (for example, Spacer C12), palmitate phosphoramidite, 3-cyanovinylcarbazole phosphoramidite, cholesteryl, and psoralen (for example, psoralen C2 phosphoramidite, and psoralen C6 phosphoramidite).

In one embodiment, the TSO comprises at least 2, 3, 4, 5, 6, 7, 8, 9, 10 or more than 10 consecutive abasic sites at the 5′ end. In some embodiments, the abasic sites prevent binding to additional nucleic acid molecules and prevents binding by the reverse transcriptase, thus the abasic sites prevent further extension of the sequence by repeated rounds of reverse transcription and template switching. Exemplary abasic sites include, but are not limited to, apurinic and apyrimidinic sites. In some embodiments, the TSO comprises 3 consecutive abasic sites at the 5′ end.

In one embodiment, the TSO comprises a nucleotide sequence to minimize base pairing between any two TSO molecules and between the TSO and the RT primer.

In one aspect, the present invention provides a composition comprising an optimized RT primer. In one embodiment, the RT primer comprises at least one 5′ end modification. In some embodiments the RT primer comprises a combination of 5′ end modifications. In some embodiments, the modification of the 5′ end comprises a bulky adduct that prevents binding of additional nucleic acid molecules to a single stranded overhang on the template molecule.

In one embodiment, the RT primer comprises at least one non-standard nucleotide. In some embodiments, at least one non-standard nucleotide is at the 5′-end of the RT primer. In one embodiment, the non-standard nucleotide is an isodeoxycytosine.

In one embodiment, the RT primer comprises at least one chemical group that blocks the 5′ end. In some embodiments, the chemical group comprises a bulky adduct that prevents binding of additional nucleic acid molecules to a single stranded overhang on the template molecule. Exemplary chemical groups that can be added to the RT primer to block the 5′ end to prevent concatemerization include, but are not limited to, 5′ trebbler, and 5′ trityl.

In one embodiment, the RT primer comprises at least 2, 3, 4, 5, 6, 7, 8, 9, 10 or more than 10 consecutive abasic sites at the 5′ end. In some embodiments, the abasic sites prevent binding to additional nucleic acid molecules and prevents binding by the reverse transcriptase, thus the abasic sites prevent further extension of the sequence by repeated rounds of reverse transcription. Exemplary abasic sites include, but are not limited to, apurinic and apyrimidinic sites. In some embodiments, the RT primer comprises 3 consecutive abasic sites at the 5′ end.

In one embodiment, the TSO and the RT primer comprise a nucleotide sequence to designed minimize base pairing between any two RT primer molecules, any two TSO molecules and between the RT primer and the TSO. In some embodiments, the sequences of the TSO and RT primer comprise only cytosine and thymidine nucleotides, which prevents the formation of base pairs between the TSO and RT primer. For example, in one embodiment, the nucleotide sequence of optimized RT primer is 5′-CCTTCTCCTTCTCCTCCTTTCTCCTTTTTTTTTTTTTTTTTT-3′ (SEQ ID NO:2); and the nucleotide sequence of optimized template switching oligo is 5′-CCCTCTCTCTCTCTTTCCTCTCTCTTTT-3′ (SEQ ID NO:3), however the invention is not limited to the exemplified RT primer and TSO.

In one aspect, the present invention provides a composition comprising an optimized RT reaction buffer.

In one aspect, the present invention provides an optimized reaction buffer that enhances the activity of the reverse transcriptase. In some embodiments, the optimized reaction buffer comprises one or more of: PEG8000 at a concentration of about 1% to about 20%, Tris at a concentration of about 10 mM to about 100 mM; LiCl at a concentration of about 20 mM to about 500 mM, MgCl₂ at a concentration of about 0.5 mM to about 5 mM, and DTT at a concentration of about 1 mM to about 10 mM. In one embodiment, the optimized reaction buffer has a pH of about 7.5 to 8.5.

In some embodiments, the optimized reaction buffer further comprises a protein stabilizing agent. Exemplary protein stabilizing agents include, but are not limited to, osmolytic stabilizers such as glycerol, erythritol, arabitol, sorbitol, mannitol, xylitol, mannisdomannitol, glucosylglycerol, glucose, fructose, sucrose, trehalose, isofluorosid, dextrans, levans, and polyethylene glycol; amino acids and derivatives thereof such as glycine, alanine, proline, taurine, betaine, octopine, glutamate, sarcosine, γ-aminobutyric acid, trimethylamine, N-oxide (TMAO); ionic stabilizers such as citrate, sulfates, acetate, phosphates, and quaternary amines; and proteins such as bovine serum albumin (BSA).

In one embodiment, the invention provides a method of performing RT comprising contacting an RNA sample with an optimized RT primer and an optimized TSO in an optimized RT reaction buffer.

In some embodiments, the reverse transcriptase is a group II intron RT. In some embodiments, the reverse transcriptase is a retroviral RT. Exemplary reverse transcriptases that can be used in the assay of the invention include, but are not limited to, MarathonRT, MMLV RT, AMV RT, HIV RT, R2 RT and TGIRT™, or a variant thereof. In some embodiments, the reverse transcription reaction efficiently creates full-length DNA products. In another embodiment, the reverse transcription reaction requires less of at least one of the TSO oligonucleotide, the RT primer, RNA template, the reverse transcriptase, or a combination thereof, relative to the amount of reverse transcriptase required in a reverse transcription reaction which uses another TSO or RT primer. In one embodiment, the method comprises amplification of RNA in a single reaction.

### Definitions

Unless defined otherwise, all technical and scientific terms used herein have the same meaning as commonly understood by one of ordinary skill in the art to which this invention belongs. It is also to be understood that the terminology used herein is for the purpose of describing particular embodiments only, and is not intended to be limiting.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

The terms “cells” and “population of cells” are used interchangeably and generally refer to a plurality of cells, i.e., more than one cell. The population may be a pure population comprising one cell type. Alternatively, the population may comprise more than one cell type. In the present invention, there is no limit on the number of cell types that a cell population may comprise.

The term “complementary” as used herein with reference to a polynucleotide is defined as having a sequence comprising the reverse complement sequence of another polynucleotide sequence, e.g., a polynucleotide having a sequence capable of base pairing (e.g. Watson-Crick base pairing) with another polynucleotide to form an antiparallel double-stranded polynucleotide duplex.

The term “end” as used herein with reference to a polynucleotide refers to any sequence that lies within the first 20 nucleotides of the polynucleotide, e.g., the 5′ end, or last 20 nucleotides of the polynucleotide, e.g., the 3′ end. In some embodiments, a template switching oligonucleotide (TSO) comprises a modification within the 5′ end of the TSO or within the 3′ end of the TSO.

A “terminus,” as used herein, refers to the first nucleotide of a polynucleotide sequence, e.g., the 5′ terminus, or the last nucleotide of a polynucleotide sequence, e.g., the 3′ terminus. In some embodiments, a template switching oligonucleotide (TSO) comprises a modification at the 5′ terminus of the TSO and/or at the 3′ terminus of the TSO.

“Encoding” refers to the inherent property of specific sequences of nucleotides in a polynucleotide, such as a gene, a DNA, or an RNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared X 100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

“Isolated” means altered or removed from the natural state. For example, a nucleic acid or a peptide naturally present in a living organism is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s). The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means. As used herein, the terms “oligonucleotide” and “polynucleotide” are used interchangeably.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a conditional manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

The term “template” as used herein, with respect to a polynucleotide, refers to a single-stranded polynucleotide substrate for a nucleic acid polymerase, e.g., a reverse transcriptase. For example, a nucleic acid polymerase, e.g., a reverse transcriptase, can synthesize a polynucleotide strand that is complementary to the template strand. In some embodiments, a single-stranded RNA polynucleotide can be a template for a reverse transcriptase.

The term “product” as used herein, with respect to a polynucleotide, refers to the polynucleotide strand synthesized by a nucleotide polymerase. In some embodiments, the nucleotide polymerase is a reverse transcriptase. In some embodiments, the product polynucleotide is a deoxyribonucleic acid (DNA) polynucleotide synthesized by a reverse transcriptase using a ribonucleic acid (RNA) polynucleotide as a template.

The term “reverse transcription” as used herein, with respect to a subject molecule, e.g., a RNA polynucleotide, refers to synthesis of a deoxyribonucleic acid (DNA), e.g., cDNA, polynucleotide using a ribonucleic acid (RNA) polynucleotide as a template.

The term “reverse transcriptase” as used herein refers a nucleic acid polymerase capable of synthesizing a deoxyribonucleic acid (DNA) polynucleotide from a template ribonucleic acid (RNA) polynucleotide. For example, a reverse transcriptase may synthesize a single-stranded complementary DNA (cDNA) polynucleotide product from a messenger RNA (mRNA) expressed in a cell or subject. In some embodiments, a reverse transcriptase may comprise a MarathonRT reverse transcriptase, a Moloney Murine Luekemia Virus reverse transcriptase, an Avian Myeloblastosis Virus reverse transcriptase, Bombyx mori R2 RNA element reverse transcriptase, or a TGIRT™ reverse transcriptase.

The term “non-templated nucleotide addition” as used herein refers to the addition of nucleotides to the 3′ end of a product polynucleotide synthesized by a reverse transcriptase upon reaching the 5′ terminus of a template polynucleotide, e.g., addition of nucleotides to the product polynucleotide that are not comprised in the template polynucleotide. For example, non-templated nucleotide addition can result in a product polynucleotide that comprises a 3′ end which extends beyond the 5′ end of the template polynucleotide and is non-complementary to the template polynucleotide. Typically, non-templated nucleotide addition results in a 1-3 nucleotide overhang, e.g., 1, 2, or 3 nucleotide overhang, at the 3′ end of the product polynucleotide relative to the template polynucleotide.

The term “template switching” as used herein refers to the process of a reverse transcriptase switching from a first template polynucleotide to a second template polynucleotide while synthesizing a continuous product polynucleotide. Typically, template switching comprises: (i) non-templated nucleotide addition of nucleotides to the 3′ end of the polynucleotide synthesized by the reverse transcriptase upon reaching the 5′ terminus of the template polynucleotide; (ii) base pairing between a template switching oligonucleotide (TSO) and the nucleotide overhang resulting from non-templated addition; and (iii) continued synthesis of the product polynucleotide by the reverse transcriptase using the TSO as the template polynucleotide. For example,

The term “concatemerization” as used herein refers to the linkage of a plurality of the same polynucleotide sequence in series, e.g., the linkage of a plurality of template switching oligonucleotide (TSO) sequences. In some embodiments, concatemerization of a plurality of a TSO can be a result of repeated cycles of non-templated nucleotide addition by a reverse transcriptase followed by template switching by the reverse transcriptase.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In some embodiments, the present invention relates to optimized compositions for performing reverse transcription and methods of use thereof. The compositions of the present invention are described herein to enhance the yield of a reverse transcription assay, to reduce concatemerization of the template switching oligonucleotide (TSO), to reduce non-specific reverse transcription from TSO molecules, and to provide sensitive and quantitative detection of RNA transcripts. The presently described TSO, RT primer and optimized reaction conditions thus provide an enhanced assay system that can be utilized in a wide variety of applications including, but not limited to, RNA sequencing, RNA amplification, next generation sequencing, nanopore sequencing, RT-PCR, quantitative PCR, cDNA synthesis, cDNA library synthesis, splice site characterization, viral RNA sequencing, single cell sequencing, RNA structure probing, and the like.

In one aspect, the present invention provides a method for reverse transcription. For example, in one embodiment, the method comprises contacting an RNA molecule with one or more TSO described herein and one or more RT primer described herein and further contacting the RNA molecule with a highly processive reverse transcriptase.

### Template Switching Oligonucleotide

In one aspect, the present invention provides a template switching oligonucleotide (TSO) that has been modified to reduce concatemerization and nonspecific reverse transcription.

The isolated TSO may be a DNA, RNA or modified oligonucleotide sequence. The isolated TSO may be a hybrid DNA/RNA oligonucleotide or modified sequence comprising 8 to 30 DNA nucleotides at the 5′ end linked to 3-8 RNA nucleotides at the 3′ end, wherein the inclusion of the RNA nucleotides promotes binding of the TSO to the DNA molecule. In some embodiments, the TSO is a hybrid DNA/RNA oligonucleotide. For example, the TSO may comprise DNA nucleotides at the 5′ and RNA nucleotides at the 3′ end. In some embodiments, the 5′ end of the TSO is 8-15 DNA nucleotides in length and the 3′ end of the TSO is 3-8 RNA nucleotides in length. In some embodiments, the 5′ end of the TSO is 15-20 DNA nucleotides in length and the 3′ end of the TSO is 3-8 RNA nucleotides in length. In some embodiments, the 5′ end of the TSO is 20-30 DNA nucleotides in length and the 3′ end of the TSO is 3-8 RNA nucleotides in length. In some embodiments, the 5′ end of the TSO is 8, 9, 10, 11, 12, 13, 14, 15, 16, 17, 18, 19, 20, 21, 22, 23, 24, 25, 26, 27, 28, 29, or 30 DNA nucleotides in length and the 3′ end of the TSO is 3, 4, 5, 6, 7, or 8 RNA nucleotides in length. In some embodiments, the TSO is a DNA oligonucleotide. In some embodiments, the TSO is 8-38 DNA nucleotides in length. In some embodiments, the TSO is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, or 38 DNA nucleotides in length.

In some embodiments, the TSO comprises at least one modified or non-natural nucleotide. In one embodiment, the 5′ end comprises at least one non-natural nucleotide or nucleotide analog, e.g., the 5′ terminus of the TSO comprises a non-natural nucleotide or nucleotide analog. In some embodiments, the TSO comprises at least one isodeoxycytosine (iso-dC), isodeoxyguanosine (iso-dG) or a combination of iso-dC and iso-dG at the 5′ end. In some embodiments, the 5′ end of the TSO comprises isodeoxycytosine (iso-dC). In some embodiments, the 5′ end of the TSO comprises isodeoxyguanosine (iso-dG). In some embodiments, the 5′ terminus of the TSO comprises isodeoxycytosine (iso-dC). In some embodiments, the 5′ terminus of the TSO comprises isodeoxyguanosine (iso-dG). In some embodiments, the 5′ end of the TSO comprises both isodeoxycytosine (iso-dC) and isodeoxyguanosine (iso-dG).

In some embodiments, the TSO comprises at least one 3′ end modification, e.g., the 3′ terminus of the TSO comprises a chemical modification. Exemplary 3′ end modifications include, but are not limited to, 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1).

In an embodiment, the 3′ terminus of the TSO comprises dideoxythymidine (ddT). In an embodiment, the 3′ terminus of the TSO comprises dideoxyuridine (ddU). In an embodiment, the 3′ terminus of the TSO comprises an inverted deoxythymidine (dT). In an embodiment, the 3′ terminus of the TSO comprises a C3 spacer. In an embodiment, the 3′ terminus of the TSO comprises an amino. In an embodiment, the 3′ terminus of the TSO comprises uridine (rU) oxidized by periodate. In an embodiment, the 3′ terminus of the TSO is phosphorylated. In an embodiment, the 3′ terminus of the TSO comprises a fluoro. In an embodiment, the 3′ terminus of the TSO comprises an aldehyde. In an embodiment, the 3′ terminus of the TSO comprises a carboxylate. In an embodiment, the 3′ terminus of the TSO comprises a thiol. In an embodiment, the 3′ terminus of the TSO comprises an O-methyl. In an embodiment, the 3′ terminus of the TSO comprises an azido. In an embodiment, the 3′ terminus of the TSO comprises an alkyne. In an embodiment, the 3′ terminus of the TSO comprises an alkene. In an embodiment, the 3′ terminus of the TSO comprises (CH2)n-X (X=H, OCH3, CH3, SH, NH2, OH, etc.), wherein n≥1. In an embodiment, the 3′ terminus of the TSO comprises (CH2CH2O)n, wherein n≥1.

In some embodiments, the TSO of the invention comprises a 5′ end modification, e.g., the 5′ terminus of the TSO comprises a chemical modification. In one embodiment, the TSO comprises at least one chemical group that blocks the 5′ end. Exemplary chemical groups that can be added to the TSO to block the 5′ end to prevent concatemerization include, but are not limited to 5′AP site (apurinic/apyrimidinic site), trityl, dendrimers (for example, trebbler), biotin, fluorescent dyes, ROX NHS ester, (CH2)n (n≥1) long spacer (for example, Spacer C12), palmitate phosphoramidite, 3-cyanovinylcarbazole phosphoramidite, cholesteryl, and psoralen (for example, psoralen C2 phosphoramidite, and psoralen C6 phosphoramidite).

In some embodiments, the 5′ terminus of the TSO comprises a trityl. In some embodiments, the 5′ terminus of the TSO comprises a dendrimer. In some embodiments, the 5′ terminus of the TSO comprises a trebbler. In some embodiments, the 5′ terminus of the TSO comprises biotin. In some embodiments, the 5′ terminus of the TSO comprises a fluorescent dye. In some embodiments, the 5′ terminus of the TSO comprises ROX NHS ester. In some embodiments, the 5′ terminus of the TSO comprises a (CH2)n long spacer, wherein n≥1. In some embodiments, the 5′ terminus of the TSO comprises a spacer C12. In some embodiments, the 5′ terminus of the TSO comprises palmitate phosphoramidite. In some embodiments, the 5′ terminus of the TSO comprises 3-cyanovinylcarbazole phosphoramidite. In some embodiments, the 5′ terminus of the TSO comprises cholesteryl. In some embodiments, the 5′ terminus of the TSO comprises psoralen. In some embodiments, the 5′ terminus of the TSO comprises psoralen C2 phosphoramidite. In some embodiments, the 5′ terminus of the TSO comprises psoralen C6 phosphoramidite. In some embodiments, the 5′ terminus of the TSO comprises an abasic site. In some embodiments, the 5′ terminus of the TSO comprises an apurinic site. In some embodiments, the 5′ terminus of the TSO comprises an apyrimidinic site. In some embodiments, the 5′ end of the TSO comprises 1-5 abasic sites. In some embodiments, the 5′ end of the TSO comprises one abasic site. In some embodiments, the 5′ end of the TSO comprises two abasic sites. In some embodiments, the 5′ end of the TSO comprises three abasic sites. In some embodiments, the 5′ end of the TSO comprises four abasic sites. In some embodiments, the 5′ end of the TSO comprises five abasic sites. In some embodiments, the 5′ end of the TSO comprises 1-5 apurinic sites. In some embodiments, the 5′ end of the TSO comprises one apurinic site. In some embodiments, the 5′ end of the TSO comprises two apurinic sites. In some embodiments, the 5′ end of the TSO comprises three apurinic sites. In some embodiments, the 5′ end of the TSO comprises four apurinic sites. In some embodiments, the 5′ end of the TSO comprises five apurinic sites. In some embodiments, the 5′ end of the TSO comprises 1-5 apyrimidinic sites. In some embodiments, the 5′ end of the TSO comprises one apyrimidinic site. In some embodiments, the 5′ end of the TSO comprises two apyrimidinic sites. In some embodiments, the 5′ end of the TSO comprises three apyrimidinic sites. In some embodiments, the 5′ end of the TSO comprises four apyrimidinic sites. In some embodiments, the 5′ end of the TSO comprises five apyrimidinic sites. In some embodiments, the TSO has the nucleotide sequence of SEQ ID NO: 3. In some embodiments, the TSO having SEQ ID NO: 3 comprises a chemical modification. In some embodiments, the 5′ terminus of the TSO having SEQ ID NO: 3 comprises a trityl. In some embodiments, the 5′ terminus of the TSO having SEQ ID NO: 3 comprises a dendrimer. In some embodiments, the 5′ terminus of the TSO having SEQ ID NO: 3 comprises a trebbler. In some embodiments, the 5′ terminus of the TSO having SEQ ID NO: 3 comprises biotin. In some embodiments, the 5′ terminus of the TSO having SEQ ID NO: 3 comprises a fluorescent dye. In some embodiments, the 5′ terminus of the TSO having SEQ ID NO: 3 comprises ROX NHS ester. In some embodiments, the 5′ terminus of the TSO having SEQ ID NO: 3 comprises a (CH2)n long spacer, wherein n≥1. In some embodiments, the 5′ terminus of the TSO having SEQ ID NO: 3 comprises a spacer C12. In some embodiments, the 5′ terminus of the TSO having SEQ ID NO: 3 comprises palmitate phosphoramidite. In some embodiments, the 5′ terminus of the TSO having SEQ ID NO: 3 comprises 3-cyanovinylcarbazole phosphoramidite. In some embodiments, the 5′ terminus of the TSO having SEQ ID NO: 3 comprises cholesteryl. In some embodiments, the 5′ terminus of the TSO having SEQ ID NO: 3 comprises psoralen. In some embodiments, the 5′ terminus of the TSO having SEQ ID NO: 3 comprises psoralen C2 phosphoramidite. In some embodiments, the 5′ terminus of the TSO having SEQ ID NO: 3 comprises psoralen C6 phosphoramidite. In some embodiments, the 5′ end of the TSO having SEQ ID NO: 3 comprises an abasic site. In some embodiments, the 5′ end of the TSO having SEQ ID NO: 3 comprises an apurinic site. In some embodiments, the 5′ end of the TSO having SEQ ID NO: 3 comprises an apyrimidinic site. In some embodiments, the 5′ end of the TSO having SEQ ID NO: 3 comprises 1-5 abasic sites. In some embodiments, the 5′ end of the TSO having SEQ ID NO: 3 comprises one abasic site. In some embodiments, the 5′ end of the TSO having SEQ ID NO: 3 comprises two abasic sites. In some embodiments, the 5′ end of the TSO having SEQ ID NO: 3 comprises three abasic sites. In some embodiments, the 5′ end of the TSO having SEQ ID NO: 3 comprises four abasic sites. In some embodiments, the 5′ end of the TSO having SEQ ID NO: 3 comprises five abasic sites. In some embodiments, the 5′ end of the TSO having SEQ ID NO: 3 comprises 1-5 apurinic sites. In some embodiments, the 5′ end of the TSO having SEQ ID NO: 3 comprises one apurinic site. In some embodiments, the 5′ end of the TSO having SEQ ID NO: 3 comprises two apurinic sites. In some embodiments, the 5′ end of the TSO having SEQ ID NO: 3 comprises three apurinic sites. In some embodiments, the 5′ end of the TSO having SEQ ID NO: 3 comprises four apurinic sites. In some embodiments, the 5′ end of the TSO having SEQ ID NO: 3 comprises five apurinic sites. In some embodiments, the 5′ end of the TSO having SEQ ID NO: 3 comprises 1-5 apyrimidinic sites. In some embodiments, the 5′ end of the TSO having SEQ ID NO: 3 comprises one apyrimidinic site. In some embodiments, the 5′ end of the TSO having SEQ ID NO: 3 comprises two apyrimidinic sites. In some embodiments, the 5′ end of the TSO having SEQ ID NO: 3 comprises three apyrimidinic sites. In some embodiments, the 5′ end of the TSO having SEQ ID NO: 3 comprises four apyrimidinic sites. In some embodiments, the 5′ end of the TSO having SEQ ID NO: 3 comprises five apyrimidinic sites.

The isolated TSO oligonucleotide can be obtained using any of the many recombinant methods known in the art, such as, for example the TSO can be produced synthetically.

The nucleic acid molecules of the present invention can be modified to improve binding to the cDNA template, reduce binding to the RT primer and TSO itself, prevent concatenation, or any combination thereof. Modifications can be added to enhance stability, functionality, and/or specificity. In some embodiments, the 3′-residue may be modified with a group that block the 3′ hydroxyl group. Exemplary modifications to block the 3′ hydroxyl group include, but are not limited to 3′ dideoxythymidine (ddT), 3′ dideoxyuridine (ddU), 3′ Inverted deoxythymidine (dT), 3′ C3 spacer, 3′ amino, 3′ uridine (rU) oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1). In an embodiment, the nucleic acid molecule comprises dideoxythymidine (ddT). In an embodiment, the nucleic acid molecule comprises dideoxyuridine (ddU). In an embodiment, the nucleic acid molecule comprises an inverted deoxythymidine (dT). In an embodiment, the nucleic acid molecule comprises a C3 spacer. In an embodiment, the nucleic acid molecule comprises an amino. In an embodiment, the nucleic acid molecule comprises uridine (rU) oxidized by periodate. In an embodiment, the nucleic acid molecule is phosphorylated. In an embodiment, the nucleic acid molecule comprises a fluoro. In an embodiment, the nucleic acid molecule comprises an aldehyde. In an embodiment, the nucleic acid molecule comprises a carboxylate. In an embodiment, the nucleic acid molecule comprises a thiol. In an embodiment, the nucleic acid molecule comprises an O-methyl. In an embodiment, the nucleic acid molecule comprises an azido. In an embodiment, the nucleic acid molecule comprises an alkyne. In an embodiment, the nucleic acid molecule comprises an alkene. In an embodiment, the nucleic acid molecule comprises (CH2)n-X (X=H, OCH3, CH3, SH, NH2, OH, etc.), wherein n≥1. In an embodiment, the nucleic acid molecule comprises (CH2CH2O)n, wherein n≥1.

In one embodiment of the present invention the nucleic acid molecule may comprise at least one modified nucleotide analogue. For example, the ends may be stabilized by incorporating modified nucleotide analogues. In some embodiments, the 5′-nucleotide may be substituted or modified with a chemical group to prevent concatemerization. Exemplary substitutions of the 5′ group to prevent concatemerization include, but are not limited to, substitution with at least one isodeoxycytosine (iso-dC), isodeoxyguanosine (iso-dG) or a combination of iso-dC and iso-dG at the 5′ end. In some embodiments, the 5′ end of the nucleic acid molecule comprises isodeoxycytosine (iso-dC). In some embodiments, the 5′ end of the nucleic acid molecule comprises isodeoxyguanosine (iso-dG). In some embodiments, the 5′ terminus of the nucleic acid molecule comprises isodeoxycytosine (iso-dC). In some embodiments, the 5′ terminus of the nucleic acid molecule comprises isodeoxyguanosine (iso-dG). In some embodiments, the 5′ end of the nucleic acid molecule comprises both isodeoxycytosine (iso-dC) and isodeoxyguanosine (iso-dG).

The TSO or RT primer of the invention may further include one or more additional nucleotide analogues. Non-limiting examples of nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides the phosphoester group connecting to adjacent ribonucleotides is replaced by a modified group, e.g., of phosphothioate group. In an embodiment, the TSO comprises a backbone modification. In an embodiment, the TSO comprises a phosphorothioate modification. In an embodiment, the RT primer comprises a backbone modification. In an embodiment, the RT primer comprises a phosphorothioate modification. In some embodiments, sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I. In an embodiment, the TSO comprises a 2′ sugar modification. In an embodiment, the TSO comprises a 2′ H. In an embodiment, the TSO comprises a 2′ O—C1-C6 alkyl. In an embodiment, the TSO comprises a 2′ O-alkenyl. In an embodiment, the TSO comprises a 2′ O-alkynyl. In an embodiment, the TSO comprises a 2′ C1-C6 alkyl. In an embodiment, the TSO comprises a 2′ alkenyl. In an embodiment, the TSO comprises a 2′ alkynyl. In an embodiment, the TSO comprises a 2′ halo. In an embodiment, the TSO comprises a 2′ F. In an embodiment, the TSO comprises a 2′ Cl. In an embodiment, the TSO comprises a 2′ Br. In an embodiment, the TSO comprises a 2′ I. In an embodiment, the TSO comprises a 2′ SH. In an embodiment, the TSO comprises a 2'S—C1-C6 alkyl. In an embodiment, the TSO comprises a 2'S-alkenyl. In an embodiment, the TSO comprises a 2'S-alkynyl. In an embodiment, the TSO comprises a 2′ NH2. In an embodiment, the TSO comprises a 2′ NH—C1-C6 alkyl. In an embodiment, the TSO comprises a 2′ NH-alkenyl. In an embodiment, the TSO comprises a 2′ NH-alkynyl. In an embodiment, the TSO comprises a 2′ N(C1-C6 alkyl)2. In an embodiment, the TSO comprises a 2′ N(alkenyl)2. In an embodiment, the TSO comprises a 2′ N(alkynyl)2. In an embodiment, the TSO comprises a 2′ ON. In an embodiment, the RT primer comprises a 2′ sugar modification. In an embodiment, the RT primer comprises a 2′ H. In an embodiment, the RT primer comprises a 2′ O—C1-C6 alkyl. In an embodiment, the RT primer comprises a 2′ O-alkenyl. In an embodiment, the RT primer comprises a 2′ O-alkynyl. In an embodiment, the RT primer comprises a 2′ C1-C6 alkyl. In an embodiment, the RT primer comprises a 2′ alkenyl. In an embodiment, the RT primer comprises a 2′ alkynyl. In an embodiment, the RT primer comprises a 2′ halo. In an embodiment, the RT primer comprises a 2′ F. In an embodiment, the RT primer comprises a 2′ Cl. In an embodiment, the RT primer comprises a 2′ Br. In an embodiment, the RT primer comprises a 2′ I. In an embodiment, the RT primer comprises a 2′ SH. In an embodiment, the RT primer comprises a 2'S—C1-C6 alkyl. In an embodiment, the RT primer comprises a 2'S-alkenyl. In an embodiment, the RT primer comprises a 2'S-alkynyl. In an embodiment, the RT primer comprises a 2′ NH2. In an embodiment, the RT primer comprises a 2′ NH—C1-C6 alkyl. In an embodiment, the RT primer comprises a 2′ NH-alkenyl. In an embodiment, the RT primer comprises a 2′ NH-alkynyl. In an embodiment, the RT primer comprises a 2′ N(C1-C6 alkyl)2. In an embodiment, the RT primer comprises a 2′ N(alkenyl)2. In an embodiment, the RT primer comprises a 2′ N(alkynyl)2. In an embodiment, the RT primer comprises a 2′ ON.

Other examples of modifications are nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine are suitable. The above modifications may be combined.

In some instances, the nucleic acid molecule comprises at least one of the following chemical modifications: 2′-H, 2′-O-methyl, or 2′-OH modification of one or more nucleotides. In certain embodiments, a nucleic acid molecule of the invention can have enhanced resistance to nucleases. For increased nuclease resistance, a nucleic acid molecule, can include, for example, 2′-modified ribose units and/or phosphorothioate linkages. For example, the 2′ hydroxyl group (OH) can be modified or replaced with a number of different “oxy” or “deoxy” substituents. For increased nuclease resistance, the nucleic acid molecules of the invention can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleobase modifications such as 2-amino-A, 2-thio (e.g., 2-thio-U), G-clamp modifications, can also increase binding affinity to a target.

In one embodiment, the nucleic acid molecule includes a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In one embodiment, the nucleic acid molecule includes at least one 2′-O-methyl-modified nucleotide, and in some embodiments, all of the nucleotides of the nucleic acid molecule include a 2′-O-methyl modification.

Nucleic acid agents discussed herein include otherwise unmodified RNA and DNA as well as RNA and DNA that have been modified. Unmodified RNA refers to a molecule in which the components of the nucleic acid, namely sugars, bases, and phosphate moieties, are the same or essentially the same as that which occur in nature. The art has referred to rare or unusual, but naturally occurring, RNAs as modified RNAs, see, e.g., Limbach et al. (Nucleic Acids Res., 1994, 22:2183-2196). Such rare or unusual RNAs, often termed modified RNAs, are typically the result of a post-transcriptional modification and are within the term unmodified RNA as used herein. Modified RNA, as used herein, refers to a molecule in which one or more of the components of the nucleic acid, namely sugars, bases, and phosphate moieties, are different from those which occur in nature. While they are referred to as “modified RNAs” they will of course, because of the modification, include molecules that are not, strictly speaking, RNAs. Nucleoside surrogates are molecules in which the ribophosphate backbone is replaced with a non-ribophosphate construct that allows the bases to be presented in the correct spatial relationship such that hybridization is substantially similar to what is seen with a ribophosphate backbone, e.g., non-charged mimics of the ribophosphate backbone.

Modifications of the nucleic acid of the invention may be present at one or more of, a phosphate group, a sugar group, backbone, N-terminus, C-terminus, or nucleobase.

### Reverse Transcription (RT) Primer

In one aspect, the present invention provides an RT primer that has been optimized to minimize duplex formation between the TSO and the RT primer and between two RT primer molecules, while retaining the ability to hybridize specifically to the RNA template molecule.

In some embodiments, the RT primer of the invention comprises a same 5′ modifications as the TSO to prevent RT primer concatemerization when the RT primer serves as a template switching oligo.

### Reverse Transcriptase

In one embodiment, the invention provides compositions and methods for use in performing reverse transcription. Reverse transcription generally refers to the process of generating a DNA molecule from an RNA template molecule and is performed by a reverse transcriptase (RT). In some embodiments, the reverse transcriptase is a group II intron RT. In some embodiments, the reverse transcriptase is a retroviral RT. Exemplary reverse transcriptases that can be used in the assay of the invention include, but are not limited to, MarathonRT, MMLV RT, AMV RT, HIV RT, R2 RT and TGIRT™.

In one embodiment, the reverse transcriptase is derived from MarathonRT. For example, in certain embodiments, the reverse transcriptase comprises MarathonRT, or a variant thereof. In one embodiment, MarathonRT is modified relative to unmodified MarathonRT. For example, in certain embodiments, the variant comprises one or more point mutations, insertion mutations, or deletion mutations, relative to wildtype MarathonRT. In certain embodiments, the variant comprises a fusion protein comprising MarathonRT, MarathonRT mutant, or MarathonRT domain.

In one embodiment, the composition comprises wildtype MarathonRT. The amino acid sequence of wildtype MarathonRT is provided below and is denoted as SEQ ID NO:1:

MDTSNLMEQILSSDNLNRAYLQVVRNKGAEGVDGMKYTELKEHLAKNGET
IKGQLRTRKYKPQPARRVEIPKPDGGVRNLGVPTVTDRFIQQAIAQVLTP
IYEEQFHDHSYGFRPNRCAQQAILTALNIMNDGNDWIVDIDLEKFFDTVN
HDKLMTLIGRTIKDGDVISIVRKYLVSGIMIDDEYEDSIVGTPQGGNLSP
LLANIMLNELDKEMEKRGLNFVRYADDCIIMVGSEMSANRVMRNISRFIE
EKLGLKVNMTKSKVDRPSGLKYLGFGFYFDPRAHQFKAKPHAKSVAKFKK
RMKELTCRSWGVSNSYKVEKLNQLIRGWINYFKIGSMKTLCKELDSRIRY
RLRMCIWKQWKTPQNQEKNLVKLGIDRNTARRVAYTGKRIAYVCNKGAVN
VAISNKRLASFGLISMLDYYIEKCVTC (E.r. maturase).

The full-length MarathonRT comprises a “secondary” RNA binding site and DNA binding domain that can influence stability, specificity, and efficiency of reverse transcription of an RNA template. In one embodiment, the reverse transcriptase comprises an MarathonRT variant where one or more secondary RNA binding sites on the surface of the protein are mutated to reduce nonspecific binding of the reverse transcription protein to the RNA template, thereby promoting binding at the polymerase cleft and facilitating enzyme turnover. In one such embodiment, a variant of MarathonRT comprises at least one point mutation selected from the group R58X, K59X, K61X, K163X, K216X, R217X, K338X, K342X, and R353X wherein X denotes any amino acid. In another such embodiment, a variant of MarathonRT comprises at least one point mutation selected from the group R58A, K59A, K61A, K163A, K216A, R217A, K338A, K342A, and R353A. Exemplary variants of MarathonRT that can be used in the reverse transcription assays of the invention include, but are not limited to, those described in detail in International Patent Publication WO2019005955A1, which is incorporated by reference herein in its entirety.

MarathonRT can perform reverse transcription at lower temperatures relative to other reverse transcriptases, and the engineering of a more thermostable MarathonRT would enable amplification of RNA templates in a single reaction (i.e., without using DNA→DNA amplification reactions). Analysis of thermophilic protein structure and function suggests that they tend to have larger numbers of side-chain hydrogen bonds and salt-bridges within rigid sections of the tertiary structure. Therefore, in one embodiment, the reverse transcriptase of the present invention comprises an MarathonRT variant, engineered to have Lys-Glu pairs at positions that are proximal in 3-D space, according to the structure of the enzyme (Zhao C et al., 2016, Nature structural & molecular biology, 23(6):558-65). In one such embodiment, the variant comprises at least one point mutation selected from the group L11E (which can form a salt bridge with R56), L21E (which can form a salt bridge with K41), and S13E (which can form a salt bridge with K52).

In one embodiment, the reverse transcriptase of the present invention comprises an MarathonRT variant, engineered to comprise a proofreading (e.g., 3′-5′ exonuclease) domain to enhance fidelity. In one such embodiment, the proofreading domain comprises an exonuclease domain. In another such embodiment, the proofreading domain is appended to the C-terminus of the MarathonRT variant. In another such embodiment, the proofreading domain is appended to the C-terminus of the MarathonRT variant through a linker molecule or sequence (see, for example, Ellefson, J W et al., 2016, Science, 352 (6293): 1590-3).

Group II intron encoded reverse transcriptases are generally conserved among species, but some may have additional, beneficial properties compared to others. Therefore, in one embodiment, the reverse transcriptase of the present invention comprises an MarathonRT variant, wherein at least one fragment or domain of MarathonRT is replaced with a fragment or domain from a group II intron encoded reverse transcriptase from a species other than Eubacterium rectale. For example, in one embodiment, the RT domain (finger and palm) of MarathonRT reverse transcriptase is replaced with the RT domain from a thermophilic group II intron encoded reverse transcriptase to enhance thermostability. In another embodiment, the α-loop of MarathonRT is replaced by a longer α-loop from another group II intron encoded reverse transcriptase to enhance processivity. In one embodiment, one or more amino acids are substituted with hydrophobic amino acids or charged amino acids in order to improve thermostability.

In one embodiment, the reverse transcriptase of the present invention comprises an MarathonRT variant, wherein one or more residues are substituted with one or more residues derived from a group II intron encoded reverse transcriptase enzyme from an organism other than Eubacterium rectale. For example, in some embodiments, the MarathonRT variant can comprise one or more point mutations based on conserved residues in thermophilic group II intron encoded reverse transcriptases. In one embodiment, the variant comprises at least one mutation selected from the group: A29X, V82X, E104X, I129X, 1137X, T161X, I168X, I170X, V171X, and M337X, where X denotes any amino acid. In one embodiment, the mutation is at least one selected from the group: A29X, V82X, E104X, I129X, I137X, T161X, I168X, I170X, V171X, and M337X, where X denotes any amino acid. In one embodiment, the variant comprises at least one mutation selected from the group: A29S, V82I, E104P, I129Y, I137V, T161R, I168L, I170L, V171I, and M337T. In one embodiment, the variant comprises a triple point mutation of A29S/V82I/E104P. In certain instances, these mutations improve upon the thermostability of the enzyme.

In one embodiment, the reverse transcriptase of the present invention comprises an MarathonRT variant, comprising one or more mutations in the thumb domain relative to wildtype MarathonRT.

In one embodiment, the variant comprises at least one point mutation selected from the group consisting of K338X, K342X, and R353X, wherein X denotes any amino acid. In another such embodiment, the variant comprises at least one point mutation selected from the group consisting of K338A, K342A, and R353A.

In one such embodiment, one or more mutations are incorporated on the surface of the thumb domain, optimizing its ability to clasp the template. In one such embodiment, the variant comprises at least one point mutation selected from the group consisting of S315X, E319X, and Q323X, wherein X denotes any amino acid. In another such embodiment, the variant comprises at least one point mutation selected from the group consisting of S315K, E319K, and Q323K.

In one embodiment, the reverse transcriptase comprises one or more mutations in the catalytic active-site to reduce the fidelity of the enzyme, which will enhance its value for RNA structure mapping since structure-specific lesions that are used to probe RNA structure are flagged by misincorporation events. Similarly, mutations that increase the error rate of the enzyme can be used with certain RNA and transcriptome mapping experiments. Therefore, in some embodiments, the polypeptide comprises at least one mutation selected from the group: A225X, R114X, Y224X, I179X, M180X, I181X, E143X, K65X, L201X, wherein X denotes any amino acid. Specifically, mutations at A225 (such as A225V, A225S, A225M or A225V), mutations at R114 (such as R114K, R114A), mutations at Y224 (such as Y224F), mutations at 1179 (such as 1179F), mutations at M180 (such as M180V), mutations at I181 (such as I181W), mutations at E143 (such as E143A or E143K), mutations at K65 (such as K65A), mutations at L201 (such as L201A or L201T), may be used, alone or in combination.

In one embodiment, the composition of the present invention comprises a polypeptide comprising Roseburia intestinalis (R.i.) maturase, or a variant or fragment thereof. In one such embodiment, the R.i. maturase comprises one or more mutations corresponding to one or more mutations described herein.

Reverse transcriptases of the present invention may produce more product (e.g., full-length product) at particular temperatures compared to other reverse transcriptases. In one aspect, comparisons of full-length product synthesis are made at different temperatures (e.g., one temperature being lower, such as between 37° C. and 50° C., and one temperature being higher, such as between 50° C. and 78° C.) while keeping all other reaction conditions similar or the same. The amount of full length product produced may be determined using techniques well known in the art, for example, by conducting a reverse transcription reaction at a first temperature (e.g., 37° C., 38° C., 39° C., 40° C., etc.) and determining the amount of full length transcript produced, conducting a second reverse transcription reaction at a temperature higher than the first temperature (e.g., 45° C., 50° C., 52.5° C., 55° C., etc.) and determining the amount of full length product produced, and comparing the amounts produced at the two temperatures. A convenient form of comparison is to determine the percentage of the amount of full-length product at the first temperature that is produced at the second (i.e., elevated) temperature. The reaction conditions used for the two reactions (e.g., salt concentration, buffer concentration, pH, divalent metal ion concentration, protein stabilizing agent concentration, macromolecular crowding agent concentration, nucleoside triphosphate concentration, template concentration, reverse transcriptase concentration, primer concentration, length of time the reaction is conducted, etc.) may be the same for both reactions. Suitable reaction conditions may be determined by those skilled in the art using routine techniques and examples of such conditions are provided herein.

The reverse transcription assay of the invention may produce at least about 5%, at least 10%, at least 15%, at least 25%, at least 50%, at least 75%, at least 100%, or at least 200% more product or full-length product compared to reverse transcription assays performed using non-optimized TSO, RT primer, or performed under different reaction conditions.

The reverse transcription assay of the invention may produce from about 2-fold to over 100-fold more product or full-length product compared to a reverse transcription assay performed under the same or different reaction conditions using a TSO with an available 3′ hydroxyl group. The reverse transcription assay of the invention may produce from about 2-fold to over 100-fold more product or full-length product compared to a reverse transcription assay performed under the same or different reaction conditions using a TSO without a 3′ end modification. The reverse transcription assay of the invention may produce from about 2-fold to over 100-fold more product or full-length product compared to a reverse transcription assay performed under the same or different reaction conditions using a TSO without non-natural nucleotide at the 5′ end. The reverse transcription assay of the invention may produce from about 2-fold to over 100-fold more product or full-length product compared to a reverse transcription assay performed under the same or different reaction conditions using a TSO without large blocking group at the 5′ end.

The reverse transcription assay of the invention may produce at least 2 times, at least 3 times, at least 4 times, at least 5 times, at least 6 times, at least 7 times, at least 8 times, at least 9 times, at least 10 times, at least 25 times, at least 50 times, at least 75 times, at least 100 times, at least 150 times, at least 200 times, at least 300 times, at least 400 times, at least 500 times, at least 1000 times, at least 5,000 times, or at least 10,000 times more product of full length product compared to a reverse transcription assay performed under the same or different reaction conditions using a TSO with an available 3′ hydroxyl group, lacking a non-natural nucleotide at the 5′ end, lacking a large chemical group at the 5′ end, or any combination thereof.

In one embodiment, the present invention provides a full-length cDNA derived from a full-length RNA, produced by a reverse transcription assay described herein. In one embodiment, the RNA has significant secondary or tertiary structure, and/or is long (greater than or equal to 5,000 bases in length). For example, MarathonRT and MarathonRT-derived peptides are highly processive reverse transcriptases. In one embodiment, the RNA reverse transcribed into DNA is at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, or at least about 10000 bases in length. In one embodiment, the DNA so reverse transcribed is at least about 100, at least about 200, at least about 300, at least about 400, at least about 500, at least about 600, at least about 700, at least about 800, at least about 900, at least about 1000, at least about 2000, at least about 3000, at least about 4000, at least about 5000, at least about 6000, at least about 7000, at least about 8000, at least about 9000, or at least about 10000 bases in length.

The invention further includes reaction solutions for reverse transcribing nucleic acid molecules, as well as reverse transcription methods employing such reaction solutions and product nucleic acid molecules produced using such methods. In many instances, reaction solutions of the invention will contain one or more of the following components: (1) one or more buffering agent (e.g., sodium phosphate, sodium acetate, 2-(N-moropholino)-ethanesulfonic acid (MES), tris-(hydroxymethyl)aminomethane (Tris), 3-(cyclohexylamino)-2-hydroxy-1-propanesulfonic acid (CAPS), citrate, N-2-hydroxyethylpiperazine-N′-2-ethanesulfonic acid (HEPES), acetate, 3-(N-morpholino) prpoanesulfonic acid (MOPS), N-tris(hydroxymethyl)methyl-3-aminopropanesulfonio acid (TAPS), etc.), (2) one or more monovalent cationic salt (e.g., LiCl, NaCl, KCl, NH₄Cl, etc.), (3) one or more divalent cationic salt (e.g., MnCl₂, MgCl₂, MgSO₄, CaCl₂, etc.), (4) one or more reducing agent (e.g., dithiothreitol, 2-mercaptoethanol, etc.), (5) one or more ionic or non-ionic detergent (e.g., TRITON X-100™, NONIDET P40™, sodium dodecyl sulphate, etc.), (6) one or more stabilizing agents (e.g., trehalose, betaine, BSA, glycerol, PEG8000) (7) one or more DNA polymerase inhibitor (e.g., Actinomycin D, etc.), (8) nucleotides (e.g., dNTPs, such as dGTP, dATP, dCTP, dTTP, etc.), (9) RNA to be reverse transcribed and/or amplified, (10) one or more RNase inhibitor (e.g., RNASEOUT™, Invitrogen Corporation, Carlsbad, Calif., etc.), (11) a reverse transcriptase, and/or (12) one or more diluent (e.g., water). Other components and/or constituents (e.g., the RT primer of the invention and TSO oligonucleotide of the invention, etc.) may also be present in reaction solutions.

In some embodiments, the invention includes an optimized reaction buffer that enhances the RT activity of MarathonRT. In one embodiment, the optimized reaction buffer comprises PEG8000 at a concentration of about 1% to 20%, Tris at a concentration of about 10 mM to about 100 mM; LiCl at a concentration of about 20 mM to about 500 mM, MgCl₂ at a concentration of about 0.5 mM to about 5 mM, and DTT at a concentration of about 1 mM to about 10 mM, and wherein the reaction buffer has a pH of about 7.5 to 8.5. In one embodiment, the optimized reaction buffer comprises about 10% PEG8000, about 50 mM Tris, about 100 mM LiCl, about 2 mM MgCl₂, about 5 mM DTT; and has a pH of about 8.3.

In one embodiment, the optimized reaction buffer further comprises a protein stabilizing agent. Exemplary protein stabilizing agents include, but are not limited to, osmolytic stabilizers such as glycerol, erythritol, arabitol, sorbitol, mannitol, xylitol, mannisdomannitol, glucosylglycerol, glucose, fructose, sucrose, trehalose, isofluorosid, dextrans, levans, and polyethylene glycol; amino acids and derivatives thereof such as glycine, alanine, proline, taurine, betaine, octopine, glutamate, sarcosine, γ-aminobutyric acid, trimethylamine, N-oxide (TMAO); ionic stabilizers such as citrate, sulfates, acetate, phosphates, and quaternary amines; and proteins such as bovine serum albumin (BSA).

In one embodiment, the optimized reaction buffer comprises trehalose at a concentration of about 0.1 M to about 1 M. In one embodiment, the optimized reaction buffer comprises betaine at a concentration of about 0.1 M to about 10 M. In one embodiment, the optimized reaction buffer comprises BSA at a concentration of about 0.5 mg/mL to about 2 mg/mL. In one embodiment, the optimized reaction buffer comprises glycerol at a concentration of about 1% to about 50%.

The concentration of the buffering agent in the reaction solutions of the invention will vary with the particular buffering agent used. Typically, the working concentration (i.e., the concentration in the reaction mixture) of the buffering agent will be from about 5 mM to about 500 mM (e.g., about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, about 100 mM, from about 5 mM to about 500 mM, from about 10 mM to about 500 mM, from about 20 mM to about 500 mM, from about 25 mM to about 500 mM, from about 30 mM to about 500 mM, from about 40 mM to about 500 mM, from about 50 mM to about 500 mM, from about 75 mM to about 500 mM, from about 100 mM to about 500 mM, from about 25 mM to about 50 mM, from about 25 mM to about 75 mM, from about 25 mM to about 100 mM, from about 25 mM to about 200 mM, from about 25 mM to about 300 mM, etc.). When Tris (e.g., Tris-HCl) is used, the Tris working concentration will typically be from about 5 mM to about 100 mM, from about 5 mM to about 75 mM, from about 10 mM to about 75 mM, from about 10 mM to about 60 mM, from about 10 mM to about 50 mM, from about 25 mM to about 50 mM, etc.

The final pH of solutions of the invention will generally be set and maintained by buffering agents present in reaction solutions of the invention. The pH of reaction solutions of the invention, and hence reaction mixtures of the invention, will vary with the particular use and the buffering agent present but will often be from about pH 5.5 to about pH 9.0 (e.g., about pH 6.0, about pH 6.5, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, from about pH 6.0 to about pH 8.5, from about pH 6.5 to about pH 8.5, from about pH 7.0 to about pH 8.5, from about pH 7.5 to about pH 8.5, from about pH 6.0 to about pH 8.0, from about pH 6.0 to about pH 7.7, from about pH 6.0 to about pH 7.5, from about pH 6.0 to about pH 7.0, from about pH 7.2 to about pH 7.7, from about pH 7.3 to about pH 7.7, from about pH 7.4 to about pH 7.6, from about pH 7.0 to about pH 7.4, from about pH 7.6 to about pH 8.0, from about pH 7.6 to about pH 8.5, from about pH 7.7 to about pH 8.5, from about pH 7.9 to about pH 8.5, from about pH 8.0 to about pH 8.5, from about pH 8.2 to about pH 8.5, from about pH 8.3 to about pH 8.5, from about pH 8.4 to about pH 8.5, from about pH 8.4 to about pH 9.0, from about pH 8.5 to about pH 9.0, etc.)

As indicated, one or more monovalent cationic salts (e.g., LiCl, NaCl, KCl, NH₄Cl, etc.) may be included in reaction solutions of the invention. In many instances, salts used in reaction solutions of the invention will dissociate in solution to generate at least one species which is monovalent (e.g., Li⁺, Na⁺, K⁺, NH₄⁺, etc.) When included in reaction solutions of the invention, salts will often be present either individually or in a combined concentration of from about 0.5 mM to about 500 mM (e.g., about 1 mM, about 2 mM, about 3 mM, about 5 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, about 55 mM, about 60 mM, about 64 mM, about 65 mM, about 70 mM, about 75 mM, about 80 mM, about 85 mM, about 90 mM, about 95 mM, about 100 mM, about 120 mM, about 140 mM, about 150 mM, about 175 mM, about 200 mM, about 225 mM, about 250 mM, about 275 mM, about 300 mM, about 325 mM, about 350 mM, about 375 mM, about 400 mM, from about 1 mM to about 500 mM, from about 5 mM to about 500 mM, from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 65 mM to about 500 mM, from about 75 mM to about 500 mM, from about 85 mM to about 500 mM, from about 90 mM to about 500 mM, from about 100 mM to about 500 mM, from about 125 mM to about 500 mM, from about 150 mM to about 500 mM, from about 200 mM to about 500 mM, from about 10 mM to about 100 mM, from about 10 mM to about 75 mM, from about 10 mM to about 50 mM, from about 20 mM to about 200 mM, from about 20 mM to about 150 mM, from about 20 mM to about 125 mM, from about 20 mM to about 100 mM, from about 20 mM to about 80 mM, from about 20 mM to about 75 mM, from about 20 mM to about 60 mM, from about 20 mM to about 50 mM, from about 30 mM to about 500 mM, from about 30 mM to about 100 mM, from about 30 mM to about 70 mM, from about 30 mM to about 50 mM, etc.).

As indicated, one or more divalent cationic salts (e.g., MnCl₂, MgCl₂, MgSO₄, CaCl₂, etc.) may be included in reaction solutions of the invention. In many instances, salts used in reaction solutions of the invention will dissociate in solution to generate at least one species which is divalent (e.g., Mg⁺⁺, Mn⁺⁺, Ca⁺⁺, etc.) When included in reaction solutions of the invention, salts will often be present either individually or in a combined concentration of from about 0.5 mM to about 500 mM (e.g., about 1 mM, about 2 mM, about 3 mM, about 4 mM, about 5 mM, about 6 mM, about 7 mM, about 8 mM, about 9 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, about 55 mM, about 60 mM, about 64 mM, about 65 mM, about 70 mM, about 75 mM, about 80 mM, about 85 mM, about 90 mM, about 95 mM, about 100 mM, about 120 mM, about 140 mM, about 150 mM, about 175 mM, about 200 mM, about 225 mM, about 250 mM, about 275 mM, about 300 mM, about 325 mM, about 350 mM, about 375 mM, about 400 mM, from about 1 mM to about 500 mM, from about 5 mM to about 500 mM, from about 10 mM to about 500 mM, from about 20 mM to about 500 mM, from about 30 mM to about 500 mM, from about 40 mM to about 500 mM, from about 50 mM to about 500 mM, from about 60 mM to about 500 mM, from about 65 mM to about 500 mM, from about 75 mM to about 500 mM, from about 85 mM to about 500 mM, from about 90 mM to about 500 mM, from about 100 mM to about 500 mM, from about 125 mM to about 500 mM, from about 150 mM to about 500 mM, from about 200 mM to about 500 mM, from about 10 mM to about 100 mM, from about 10 mM to about 75 mM, from about 10 mM to about 50 mM, from about 20 mM to about 200 mM, from about 20 mM to about 150 mM, from about 20 mM to about 125 mM, from about 20 mM to about 100 mM, from about 20 mM to about 80 mM, from about 20 mM to about 75 mM, from about 20 mM to about 60 mM, from about 20 mM to about 50 mM, from about 30 mM to about 500 mM, from about 30 mM to about 100 mM, from about 30 mM to about 70 mM, from about 30 mM to about 50 mM, etc.).

When included in reaction solutions of the invention, reducing agents (e.g., dithiothreitol, β-mercaptoethanol, etc.) will often be present either individually or in a combined concentration of from about 0.1 mM to about 50 mM (e.g., about 0.2 mM, about 0.3 mM, about 0.5 mM, about 0.7 mM, about 0.9 mM, about 1 mM, about 2 mM, about 3 mM, about 4 mM, about 5 mM, about 6 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, from about 5.0 mM to about 10 mM, from about 5.0 mM to about 15 mM, from about 5.0 mM to about 20 mM, from about 10 mM to about 15 mM, from about 10 mM to about 20 mM, etc.).

Reaction solutions of the invention may also contain one or more ionic or non-ionic detergent (e.g., TRITON X-100™, NONIDET P40™, sodium dodecyl sulfate, etc.). When included in reaction solutions of the invention, detergents will often be present either individually or in a combined concentration of from about 0.01% to about 5.0% (e.g., about 0.01%, about 0.02%, about 0.03%, about 0.04%, about 0.05%, about 0.06%, about 0.07%, about 0.08%, about 0.09%, about 0.1%, about 0.15%, about 0.2%, about 0.3%, about 0.5%, about 0.7%, about 0.9%, about 1%, about 2%, about 3%, about 4%, about 5%, from about 0.01% to about 5.0%, from about 0.01% to about 4.0%, from about 0.01% to about 3.0%, from about 0.01% to about 2.0%, from about 0.01% to about 1.0%, from about 0.05% to about 5.0%, from about 0.05% to about 3.0%, from about 0.05% to about 2.0%, from about 0.05% to about 1.0%, from about 0.1% to about 5.0%, from about 0.1% to about 4.0%, from about 0.1% to about 3.0%, from about 0.1% to about 2.0%, from about 0.1% to about 1.0%, from about 0.1% to about 0.5%, etc.). For example, reaction solutions of the invention may contain TRITON X-100™ at a concentration of from about 0.01% to about 2.0%, from about 0.03% to about 1.0%, from about 0.04% to about 1.0%, from about 0.05% to about 0.5%, from about 0.04% to about 0.6%, from about 0.04% to about 0.3%, etc.

Reaction solutions of the invention may also contain one or more stabilizing agents (e.g., PEG8000, trehalose, betaine, BSA, glycerol). In some embodiments, when included in reaction solutions of the invention, stabilizing agents are present either individually or in a combined concentration from 0.01 M to about 50 M (e.g., about 0.05M, about 0.1 M, 0.2 M, about 0.3 M, about 0.5 M, about 0.6 M, about 0.7 M, about 0.9 M, about 1 M, about 2 M, about 3 M, about 4 M, about 5 M, about 6 M, about 10 M, about 12 M, about 15 M, about 17 M, about 20 M, about 22 M, about 23 M, about 24 M, about 25 M, about 27 M, about 30 M, about 35 M, about 40 M, about 45 M, about 50 M, from about 0.1 M to about 1 M, from about 0.5 M to about 5 M, from about 0.2 M to about 2 M, from about 0.3 M to about 3 M, from about 0.4 M to about 4 M, from about 0.5 M to about 5 M, from about 0.2 M to about 0.8 M, from about 0.5 M to about 1 M, from about 0.05 M to about 1 M, from about 0.05 M to about 10 M, from about 0.05 M to about 20M, etc.). In some embodiments, when included in reaction solutions of the invention, such stabilizing agents are present either individually or in a combined concentration of from about 0.01 mg/ml to about 100 mg/ml (e.g., about 0.01 mg/ml, about 0.02 mg/ml, about 0.03 mg/ml, about 0.04 mg/ml, about 0.05 mg/ml, about 0.06 mg/ml, about 0.07 mg/ml, about 0.08 mg/ml, about 0.09 mg/ml, about 0.1 mg/ml, about 0.11 mg/ml, about 0.12 mg/ml, about 0.15 mg/ml, about 0.17 mg/ml, about 0.2 mg/ml, about 0.25 mg/ml, about 0.35 mg/ml, about 0.5 mg/ml, about 0.75 mg/ml, about 1.0 mg/ml, about 1.5 mg/ml, about 2.0 mg/ml, about 2.5 mg/ml, about 3.0 mg/ml, about 3.5 mg/ml, about 4.0 mg/ml, about 5.0 mg/ml, about 6.0 mg/ml, about 7.0 mg/ml, about 8.0 mg/ml, about 9.0 mg/ml, about 10.0 mg/ml, from about 0.05 mg/ml to about 3.0 mg/ml, from about 0.1 mg/ml to about 5.0 mg/ml, from about 0.2 mg/ml to about 2.0 mg/ml, etc.). In some embodiments, when included in reaction solutions of the invention, such stabilizing agents are be present either individually or in a combined concentration of from about 0.1% to about 50% (e.g., about 0.1%, about 0.2%, about 0.3%, about 0.4%, about 0.5%, about 0.6%, about 0.7%, about 0.8%, about 0.9%, about 1.0%, about 1.5%, about 2.0%, about 3.0%, about 5.0%, about 7.0%, about 9.0%, about 10%, about 11%, about 12%, about 13%, about 14%, about 15%, about 20%, about 22%, about 25%, about 27%, about 30%, about 35%, about 40%, about 45%, about 50%, from about 0.1% to about 50%, from about 0.1% to about 40%, from about 0.1% to about 30%, from about 0.0% to about 20%, from about 0.1% to about 10%, etc.

Reaction solutions of the invention may also contain one or more DNA polymerase inhibitor (e.g., Actinomycin D, etc.). When included in reaction solutions of the invention, such inhibitors will often be present either individually or in a combined concentration of from about 0.1 μg/ml to about 100 μg/ml (e.g., about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μg/ml, about 0.9 μg/ml, about 1.0 μg/ml, about 1.1 μg/ml, about 1.3 μg/ml, about 1.5 μg/ml, about 1.7 μg/ml, about 2.0 μg/ml, about 2.5 μg/ml, about 3.5 μg/ml, about 5.0 μg/ml, about 7.5 μg/ml, about 10 μg/ml, about 15 μg/ml, about 20 μg/ml, about 25 μg/ml, about 30 μg/ml, about 35 μg/ml, about 40 μg/ml, about 50 μg/ml, about 60 μg/ml, about 70 μg/ml, about 80 μg/ml, about 90 μg/ml, about 100 μg/ml, from about 0.5 μg/ml to about 30 μg/ml, from about 0.75 μg/ml to about 30 μg/ml, from about 1.0 μg/ml to about 30 μg/ml, from about 2.0 μg/ml to about 30 μg/ml, from about 3.0 μg/ml to about 30 μg/ml, from about 4.0 μg/ml to about 30 g/ml, from about 5.0 μg/ml to about 30 μg/ml, from about 7.5 μg/ml to about 30 μg/ml, from about 10 μg/ml to about 30 μg/ml, from about 15 μg/ml to about 30 μg/ml, from about 0.5 g/ml to about 20 μg/ml, from about 0.5 μg/ml to about 10 μg/ml, from about 0.5 μg/ml to about 5 μg/ml, from about 0.5 μg/ml to about 2 μg/ml, from about 0.5 μg/ml to about 1 μg/ml, from about 1 μg/ml to about 10 μg/ml, from about 1 μg/ml to about 5 μg/ml, from about 1 μg/ml to about 2 μg/ml, from about 1 μg/ml to about 100 μg/ml, from about 10 μg/ml to about 100 μg/ml, from about 20 μg/ml to about 100 μg/ml, from about 40 μg/ml to about 100 μg/ml, from about 30 μg/ml to about 80 μg/ml, from about 30 μg/ml to about 70 μg/ml, from about 40 μg/ml to about 60 μg/ml, from about 40 μg/ml to about 70 μg/ml, from about 40 μg/ml to about 80 μg/ml, etc.).

Reaction solutions the invention may also contain one or more additional additives that improve RT activity, including agents that improve primer utilization efficiency and improve product yield. In one embodiment, the reaction solution comprises an agent that reduces non-specific binding of primers to the MarathonRT surface. The agent may comprise any protein, nucleic acid molecule, or small molecule that prevents or reduces non-specific binding. In certain embodiments, the agent comprises D4A or variant thereof. D4A and variants of D4A that can be included in the reverse transcription assay of the invention include, but are not limited to, those described in detail in International Patent Publication WO2019005955A1, which is incorporated by reference herein in its entirety.

When included in reaction solutions of the invention, D4A, or variant thereof, may be present at ratio of D4A (or variant thereof) concentration to MarathonRT concentration from about 0.1:1 to about 100:1. For example, in some embodiments, D4A, or variant thereof, may be present at ratio of D4A (or variant thereof) concentration to MarathonRT concentration of about 0.1:1, 0.2:1, 0.3:1, 0.4:1, 0.5:1, 0.6:1, 0.7:1, 0.8:1, 0.9:1, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 55:1, 60:1, 65:1, 70:1, 75:1, 80:1, 85:1, 90:1, 95:1, or 100:1.

In many instances, nucleotides (e.g., dNTPs, such as dGTP, dATP, dCTP, dTTP, etc.) will be present in reaction mixtures of the invention. Typically, individual nucleotides will be present in concentrations of from about 0.05 mM to about 50 mM (e.g., about 0.07 mM, about 0.1 mM, about 0.15 mM, about 0.18 mM, about 0.2 mM, about 0.3 mM, about 0.5 mM, about 0.7 mM, about 0.9 mM, about 1 mM, about 2 mM, about 3 mM, about 4 mM, about 5 mM, about 6 mM, about 10 mM, about 12 mM, about 15 mM, about 17 mM, about 20 mM, about 22 mM, about 23 mM, about 24 mM, about 25 mM, about 27 mM, about 30 mM, about 35 mM, about 40 mM, about 45 mM, about 50 mM, from about 0.1 mM to about 50 mM, from about 0.5 mM to about 50 mM, from about 1 mM to about 50 mM, from about 2 mM to about 50 mM, from about 3 mM to about 50 mM, from about 0.5 mM to about 20 mM, from about 0.5 mM to about 10 mM, from about 0.5 mM to about 5 mM, from about 0.5 mM to about 2.5 mM, from about 1 mM to about 20 mM, from about 1 mM to about 10 mM, from about 1 mM to about 5 mM, from about 1 mM to about 3.4 mM, from about 0.5 mM to about 3.0 mM, from about 1 mM to about 3.0 mM, from about 1.5 mM to about 3.0 mM, from about 2 mM to about 3.0 mM, from about 0.5 mM to about 2.5 mM, from about 1 mM to about 2.5 mM, from about 1.5 mM to about 2.5 mM, from about 2 mM to about 3.0 mM, from about 2.5 mM to about 3.0 mM, from about 0.5 mM to about 2 mM, from about 0.5 mM to about 1.5 mM, from about 0.5 mM to about 1.1 mM, from about 5.0 mM to about 10 mM, from about 5.0 mM to about 15 mM, from about 5.0 mM to about 20 mM, from about 10 mM to about 15 mM, from about 10 mM to about 20 mM, etc.). The combined nucleotide concentration, when more than one nucleotide is present, can be determined by adding the concentrations of the individual nucleotides together. When more than one nucleotide is present in reaction solutions of the invention, the individual nucleotides may not be present in equimolar amounts. Thus, a reaction solution may contain, for example, 1 mM dGTP, 1 mM dATP, 0.5 mM dCTP, and 1 mM dTTP.

RNA will typically be present in reaction solutions of the invention. In most instances, RNA will be added to the reaction solution shortly prior to reverse transcription. Thus, reaction solutions may be provided without RNA. This will typically be the case when reaction solutions are provided in kits. RNA, when present in reaction solutions will often be present in a concentration of 0.01 picogram to 100 μg/20 μl reaction mixture (e.g., about 0.01 picogram/20 μl, about 0.1 picogram/20 μl, about 0.5 picogram/20 μl, about 1 picogram/20 μl, about 10 picograms/20 μl, about 50 picograms/20 μl, about 100 picograms/20 μl, about 200 picograms/20 μl, about 10 picograms/20 μl, about 500 picograms/20 μl, about 800 picograms/20 μl, about 1.0 nanogram/20 μl, about 5.0 nanograms/20 μl, about 10 nanograms/20 μl, about 25 nanograms/20 μl, about 50 nanograms/20 μl, about 75 nanograms/20 μl, about 100 nanograms/20 μl, about 150 nanograms/20 μl, about 250 nanograms/20 μl, about 400 nanograms/20 μl, about 500 nanograms/20 μl, about 750 nanograms/20 μl, about 1.0 μg/20 μl, about 5.0 μg/20 μl, about 10 μg/20 μl, about 20 μg/20 μl, about 30 μg/20 μl, about 40 μg/20 μl, about 50 μg/20 μl, about 70 μg/20 μl, about 85 μg/20 μl, about 100 μg/20 μl, from about 10 picograms/20 μl to about 100 μg/20 μl, from about 10 picograms/20 μl to about 100 μg/20 μl, from about 100 picograms/20 μl to about 100 μg/20 μl, from about 1.0 nanograms/20 μl to about 100 μg/20 μl, from about 100 nanograms/20 μl to about 100 μg/20 μl, from about 10 picograms/20 μl to about 10 μg/20 μl, from about 10 picograms/20 μl to about 5 μg/20 μl, from about 100 nanograms/20 μl to about 5 μg/20 μl, from about 1 μg/20 μl to about 10 μg/20 μl, from about 1 μg/20 μl to about 5 μg/20 μl, from about 100 nanograms/20 μl to about 1 μg/20 μl, from about 500 nanograms/20 μl to about 5 μg/20 μl, etc.). As one skilled in the art would recognize, different reverse transcription reactions may be performed in volumes other than 20 μl. In such instances, the total amount of RNA present will vary with the volume used. Thus, the above amounts are provided as examples of the amount of RNA/20 μl of reaction solution.

Reverse transcriptases may also be present in reaction solutions. When present, reverse transcriptases, will often be present in a concentration which results in about 0.01 to about 1,000 units of reverse transcriptase activity/μl (e.g., about 0.01 unit/μl, about 0.05 unit/μl, about 0.1 unit/μl, about 0.2 unit/μl, about 0.3 unit/μl, about 0.4 unit/μl, about 0.5 unit/μl, about 0.7 unit/μl, about 1.0 unit/μl, about 1.5 unit/μl, about 2.0 unit/μl, about 2.5 unit/μl, about 5.0 unit/μl, about 7.5 unit/μl, about 10 unit/μl, about 20 unit/μl, about 25 unit/μl, about 50 unit/μl, about 100 unit/μl, about 150 unit/μl, about 200 unit/μl, about 250 unit/μl, about 350 unit/μl, about 500 unit/μl, about 750 unit/μl, about 1,000 unit/μl, from about 0.1 unit/μl to about 1,000 unit/μl, from about 0.2 unit/μl to about 1,000 unit/μl, from about 1.0 unit/μl to about 1,000 unit/μl, from about 5.0 unit/μl to about 1,000 unit/μl, from about 10 unit/μl to about 1,000 unit/μl, from about 20 unit/μl to about 1,000 unit/μl, from about 50 unit/μl to about 1,000 unit/μl, from about 100 unit/μl to about 1,000 unit/μl, from about 200 unit/μl to about 1,000 unit/μl, from about 400 unit/μl to about 1,000 unit/μl, from about 500 unit/μl to about 1,000 unit/μl, from about 0.1 unit/μl to about 300 unit/μl, from about 0.1 unit/μl to about 200 unit/μl, from about 0.1 unit/μl to about 100 unit/μl, from about 0.1 unit/μl to about 50 unit/μl, from about 0.1 unit/μl to about 10 unit/μl, from about 0.1 unit/μl to about 5.0 unit/μl, from about 0.1 unit/μl to about 1.0 unit/μl, from about 0.2 unit/μl to about 0.5 unit/μl, etc. In certain embodiments, the reaction solution comprises a lower concentration of the reverse transcriptase described herein, as compared to what would be necessary to produce equivalent product from other reverse transcriptases.

Reaction solutions of the invention may be prepared as concentrated solutions (e.g., 5× solutions) which are diluted to a working concentration for final use. With respect to a 5× reaction solution, a 5:1 dilution is required to bring such a 5× solution to a working concentration. Reaction solutions of the invention may be prepared, for examples, as a 2×, a 3×, a 4×, a 5×, a 6×, a 7×, a 8×, a 9×, a 10×, etc. solutions. One major limitation on the fold concentration of such solutions is that, when compounds reach particular concentrations in solution, precipitation occurs. Thus, concentrated reaction solutions will generally be prepared such that the concentrations of the various components are low enough so that precipitation of buffer components will not occur. As one skilled in the art would recognize, the upper limit of concentration which is feasible for each solution will vary with the particular solution and the components present.

In many instances, reaction solutions of the invention will be provided in sterile form. Sterilization may be performed on the individual components of reaction solutions prior to mixing or on reaction solutions after they are prepared. Sterilization of such solutions may be performed by any suitable means including autoclaving or ultrafiltration.

### Methods

In various embodiments, the present invention includes methods of using a TSO, a RT primer or a combination of TSO and RT primer for a reverse transcription reaction. For example, in one embodiment, the method comprises contacting an RNA template with a TSO, RT primer and reverse trascriptase under suitable conditions to produce a transcribed DNA molecule from the RNA template.

In various embodiments, the present invention includes methods of performing a reverse transcription reaction using MarathonRT, or a variant thereof, or a nucleic acid encoding MarathonRT or a variant thereof; in combination with the TSO and RT primer of the invention. For example, in some embodiments, the method comprises using MarathonRT, or a variant thereof, or a nucleic acid encoding MarathonRT or a variant thereof; in combination with a TSO and an RT primer wherein the TSO comprises at least one modification of the 5′ end, at least one modification of the 3′ end, or modification of both the 5′ and 3′ end to prevent concatemerization and non-specific reverse transcription during template switching.

For example, in one embodiment, the method comprises mixing the TSO, the RT primer and a reverse transcriptase, comprising an MarathonRT or variant thereof, under suitable conditions; and contacting the mixture to an RNA template to produce a transcribed DNA molecule from the RNA template.

In various embodiments, the present invention includes methods of performing the reverse transcription in an optimized reaction buffer. For example, in one embodiment, the method comprises adding a TSO, an RT primer and a reverse transcriptase (e.g., MarathonRT or variant thereof) to an optimized reaction buffer; and contacting the mixture to an RNA template to produce a transcribed DNA molecule from the RNA template. In one embodiment, the optimized reaction buffer comprises PEG8000 at a concentration of about 1% to about 20%, Tris at a concentration of about 10 mM to about 100 mM; LiCl at a concentration of about 20 mM to about 500 mM, MgCl₂ at a concentration of about 0.5 mM to about 5 mM, DTT at a concentration of about 1 mM to about 10 mM, and wherein the reaction buffer has a pH of about 7.5 to 8.5. In one embodiment, the optimized reaction buffer comprises about 10% PEG8000, about 50 mM Tris, about 100 mM LiCl, about 2 mM MgCl₂, about 5 mM DTT; and has a pH of about 8.3.

In one embodiment the optimized reaction buffer comprises a protein stabilizing agent. Exemplary protein stabilizing agents include, but are not limited to, osmolytic stabilizers such as glycerol, erythritol, arabitol, sorbitol, mannitol, xylitol, mannisdomannitol, glucosylglycerol, glucose, fructose, sucrose, trehalose, isofluorosid, dextrans, levans, and polyethylene glycol; amino acids and derivatives thereof such as glycine, alanine, proline, taurine, betaine, octopine, glutamate, sarcosine, γ-aminobutyric acid, trimethylamine, N-oxide (TMAO); ionic stabilizers such as citrate, sulfates, acetate, phosphates, and quaternary amines; and proteins such as bovine serum albumin (BSA).

In one embodiment, the optimized reaction buffer comprises trehalose at a concentration of about 0.1 M to about 1 M. In one embodiment, the optimized reaction buffer comprises betaine at a concentration of about 0.1 M to about 10 M. In one embodiment, the optimized reaction buffer comprises BSA at a concentration of about 0.5 mg/mL to about 2 mg/mL. In one embodiment, the optimized reaction buffer comprises glycerol at a concentration of about 1% to about 50%.

### Using the TSO and RT Primer

Any technology that employs reverse transcription as a method or step can utilize the TSO, the RT primer, or a combination thereof, of the present invention. In various embodiments, the improved TSO, RT primer, or a combination thereof are used to perform reverse transcription as part of an assay. In various embodiments, the assay may be at least one selected from the group RT-PCR, qRT-PCR, capillary electrophoresis (CE) for RNA-structure mapping (such as SHAPE-seq or SHAPE-MaP, DMS-seq), transcriptome profiling, in-cell sequencing, next-generation RNA sequencing (RNA-seq), nanopore sequencing, PacBio sequencing, zero-mode waveguide sequencing, cDNA library synthesis, cDNA synthesis, and a combination thereof.

In certain aspects, the method provides for reverse transcription at physiologic temperatures, or at lower temperatures relative to that required when using non-MarathonRT-derived reverse transcriptases. In certain instances, the lower temperature of the reverse transcription reaction provides a decreased rate of degradation of the RNA molecule during the reaction, relative to the rate of degradation of an RNA molecule in a reverse transcription reaction that uses a non-MarathonRT-derived reverse transcriptase.

In one embodiment, the method comprises reverse transcription of a long and/or complex RNA molecule.

In one embodiment, the method comprises formulating a reaction solution comprising a low concentration of a TSO or RT primer described herein, compared to the concentration required for a reaction using a different TSO or RT primer.

In one embodiment, the method comprises formulating a reaction solution comprising a high concentration of a TSO or RT primer described herein, compared to the concentration required for a reaction using a different TSO or RT primer.

In one embodiment, the method comprises a single reaction amplification of RNA, made possible by the true thermocycling ability of the reverse transcriptases described herein. For, example, the thermocycling ability of the reverse transcriptases described herein allows for the amplification of RNA without the need for DNA replication.

In one embodiment, the improved TSO, RT primer, or a combination thereof is utilized in a quantitative RT-PCR (qRT-PCR) procedure. In qRT-PCR, the formation of PCR products is monitored in each cycle of the PCR. The amplification is usually measured in thermocyclers which have additional devices for measuring fluorescence signals during the amplification reaction. See, for example, U.S. Pat. Nos. 6,174,670, and 8,137,616. In one embodiment, the qRT-PCR procedure is carried out using a thermostable improved MarathonRT enzyme, without a DNA→DNA polymerase.

In one embodiment, the improved TSO, RT primer, or a combination thereof is utilized in isothermal DNA amplification using an engineered reverse transcriptase with improved stand-displacement activity on DNA templates.

In one embodiment, the improved TSO, RT primer, or a combination thereof is utilized in a capillary electrophoresis (CE) for RNA-structure mapping procedure. The application of capillary electrophoresis to RNA structure probing is an important step in increasing the throughput of RNA structure data. Gel electrophoresis typically resolves about a hundred bases of RNA at a time, and hence probing an RNA of several kilobases long might require running tens to hundreds of gels. Capillary electrophoresis allows the resolution of 300-650 bases from a structure probing experiment and multiple lanes can be run at the same time to increase the throughput of RNA structure probing. The readout of the probing experiment is typically through the reverse transcription of a 5′ fluorescently labeled DNA primer that anneals specifically to the RNA of interest. If the RNA is several kilobases long, multiple primers are designed to anneal along the length of the transcript. Modification or cleavage of the RNA template results in premature stops in the primer extension reaction, leading to different lengths of the cDNA product which are resolved by capillary electrophoresis. Software tools such as CAFA and Shapefinder can automate the data acquisition from capillary electrophoresis and further improve speed and accuracy (see, for example, Wan, Y. et al., 2011, Nat Rev Genet., 12(9):1-26).

In one embodiment, the improved TSO, RT primer, or a combination thereof is utilized in a next-generation RNA sequencing (RNA-seq) procedure. High-throughput RNA sequencing (RNA-Seq) technology, enabled by recent developments in next generation sequencing, has become a powerful tool in analyzing gene expression profiles, detecting transcript variants, and understanding the function of non-coding regulatory RNAs. A standard RNA-Seq library is generated from ligating sequencing adapters to double-stranded DNA. There are two main classes of methods to prepare strand-specific RNA-Seq libraries. The first method comprises ligating different adapters to the 3′ and 5′ ends of the RNA molecules (see e.g. Ion Total RNA-Seq Kit v2 from Life Technologies). Another, more widely used method comprises incorporating dUTP in addition to dNTPs in the second strand DNA synthesis. Following adapter ligation, the second strand DNA can be specifically digested by an Uracil-N-glycosylase (UNG) enzyme so that only the library strand containing the first strand cDNA will be sequenced and information on the direction of the transcripts can therefore be obtained (see M. Sultan et al., Biochemical and Biophysical Research Communications 422 (2012) 643-646; also see PCT Patent Application Number PCT/EP2016/069997).

The invention is also directed to methods for making one or more nucleic acid molecules and/or labeled nucleic acid molecules, comprising mixing one or more nucleic acid templates (e.g., one or more RNA templates or messenger RNA templates) with a TSO, RT primer, or a combination thereof and one or more polypeptides having reverse transcriptase activity and incubating the mixture under conditions sufficient to synthesize one or more first nucleic acid molecules complementary to all or a portion of the one or more nucleic acid templates, wherein at least one of the synthesized molecules are optionally labeled and/or comprise one or more labeled nucleotides and/or wherein the synthesized molecules may optionally be modified to contain one or more labels. In one embodiment, the one or more first nucleic acid molecules are single-stranded cDNA molecules. Nucleic acid templates suitable for reverse transcription according to this aspect of the invention include any nucleic acid molecule or population of nucleic acid molecules (e.g., RNA, mRNA), particularly those derived from a cell or tissue. In one aspect, a population of mRNA molecules (a number of different mRNA molecules, typically obtained from cells or tissue) are used to make a labeled cDNA library, in accordance with the invention. Exemplary sources of nucleic acid templates include viruses, virally infected cells, bacterial cells, fungal cells, plant cells and animal cells.

The invention also concerns methods for making one or more double-stranded nucleic acid molecules (which may optionally be labeled). Such methods comprise (a) mixing one or more nucleic acid templates (e.g., RNA or mRNA, or a population of mRNA templates) with a TSO, RT primer, or a combination thereof and one or more polypeptides having reverse transcriptase activity; (b) incubating the mixture under conditions sufficient to make one or more first nucleic acid molecules complementary to all or a portion of the one or more templates; and (c) incubating the one or more first nucleic acid molecules under conditions sufficient to make one or more second nucleic acid molecules complementary to all or a portion of the one or more first nucleic acid molecules, thereby forming one or more double-stranded nucleic acid molecules comprising the first and second nucleic acid molecules. In accordance with the invention, the first and/or second nucleic acid molecules may be labeled (e.g., may comprise one or more of the same or different labeled nucleotides and/or may be modified to contain one or more of the same or different labels). Thus, labeled nucleotides may be used at one or both synthesis steps. Such methods may include the use of one or more DNA polymerases as part of the process of making the one or more double-stranded nucleic acid molecules. The invention also concerns compositions useful for making such double-stranded nucleic acid molecules. Such compositions comprise TSO, RT primer, or a combination thereof, one or more reverse transcriptases and optionally one or more DNA polymerases, a suitable buffer and/or one or more nucleotides (e.g., including labeled nucleotides).

The invention is also directed to nucleic acid molecules and/or labeled nucleic acid molecules (particularly single- or double-stranded cDNA molecules) produced according to the above-described methods and to kits comprising these nucleic acid molecules. Such molecules or kits may be used to detect nucleic acid molecules (for example by hybridization) or for diagnostic purposes.

### Kits

The invention is also directed to kits for use in the reverse transcription methods of the invention. Such kits can be used for making nucleic acid molecules and/or labeled nucleic acid molecules (single- or double-stranded). Kits of the invention may comprise a carrier, such as a box or carton, having in close confinement therein one or more containers, such as vials, tubes, bottles and the like. In kits of the invention, a first container may contain one or more of the reverse transcriptase enzymes of the invention or one or more of the compositions of the invention. Kits of the invention may also comprise, in the same or different containers, at least one component selected from one or more TSO, one or more RT primer, and a reverse transcriptase. In one embodiment, kits of the invention may also comprise, in the same or different containers, an agent that reduces non-specific binding of primers to the reverse transcriptase. In one embodiment, kits of the invention may also comprise, in the same or different containers, an optimized reaction buffer as described elsewhere herein, or components used to produce the optimized reaction buffer. Alternatively, the components of the kit may be divided into separate containers.

The invention is also directed to kits for use in methods of the invention. Such kits can be used for making, sequencing or amplifying nucleic acid molecules (single- or double-stranded), e.g., at the particular temperatures described herein. Kits of the invention may comprise a carrier, such as a box or carton, having in close confinement therein one or more (e.g., one, two, three, four, five, ten, twelve, fifteen, etc.) containers, such as vials, tubes, bottles and the like. In kits of the invention, a first container contains one or more of the reverse transcriptase enzymes of the present invention. Kits of the invention may also comprise, in the same or different containers, one or more DNA polymerases (e.g., thermostable DNA polymerases), one or more (e.g., one, two, three, four, five, ten, twelve, fifteen, etc.) suitable buffers for nucleic acid synthesis, one or more nucleotides and one or more (e.g., one, two, three, four, five, ten, twelve, fifteen, etc.) oligonucleotide primers. Kits of the invention also may comprise instructions or protocols for carrying out the methods of the invention.

In various embodiments, the present invention provides a kit for use in performing a reverse transcription reaction. In one embodiment, the kit comprises at least one TSO oligonucleotide, at least one RT primer and a reverse transcription polypeptide or a variant thereof. In one embodiment, the kit includes instructional material that describes the use of the kit to perform a reverse transcription reaction, wherein the instructional material creates an increased functional relationship between the kit components and the individual using the kit. In one embodiment, the kit is utilized by one person or entity. In another embodiment, the kit is utilized by more than one person or entity. In one embodiment, the kit is used without any additional compositions or methods. In another embodiment, the kit is used with at least one additional composition or method.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Optimization of Template Switching Oligonucleotides (TSOs)

In an RNA-seq experiment, accurate quantification of individual transcripts, identification of novel transcripts or identification of unknown transcription start sites requires an efficient approach to convert mRNA molecules into full-length cDNA. Meanwhile, both 5′- and 3′-end of cDNA molecules need to be efficiently attached with two different adapters of universal sequences for library preparation. These steps can be achieved simultaneously by combining the highly processive reverse transcriptase activity and template switching activity of MarathonRT (or E.r. maturase) in a one-pot reaction, which is particularly useful when the amount of RNA input is very low such as that from a single cell. The template switching oligos (TSOs) that contain universal sequences of choice are designed to be attached to the 3′-end of cDNA molecules during template switching.

Specifically, during first-strand synthesis, upon reaching the 5′ end of the RNA template (such as cellular RNA), the terminal transferase activity of MarathonRT adds a few additional nucleotides (mostly deoxyadenosine) to the 3′ end of the newly synthesized cDNA strand in a non-templated fashion. These bases function as a TSO-anchoring site during template switching. Upon base pairing between the TSO and the appended deoxyadenosine stretch, MarathonRT “switches” template strands seamlessly, from cellular RNA to the TSO, and continues primer extension to the 5′ end of the TSO. By doing so, the resulting cDNA contains the complete 5′ end of the transcript, and universal sequences of choice are added to the reverse transcription product. Along with tagging of the cDNA 5′ end by oligo dT primers, this approach makes it possible to efficiently amplify the entire full-length transcript pool in a completely sequence-independent manner.

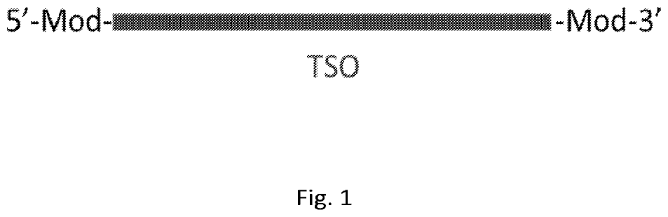

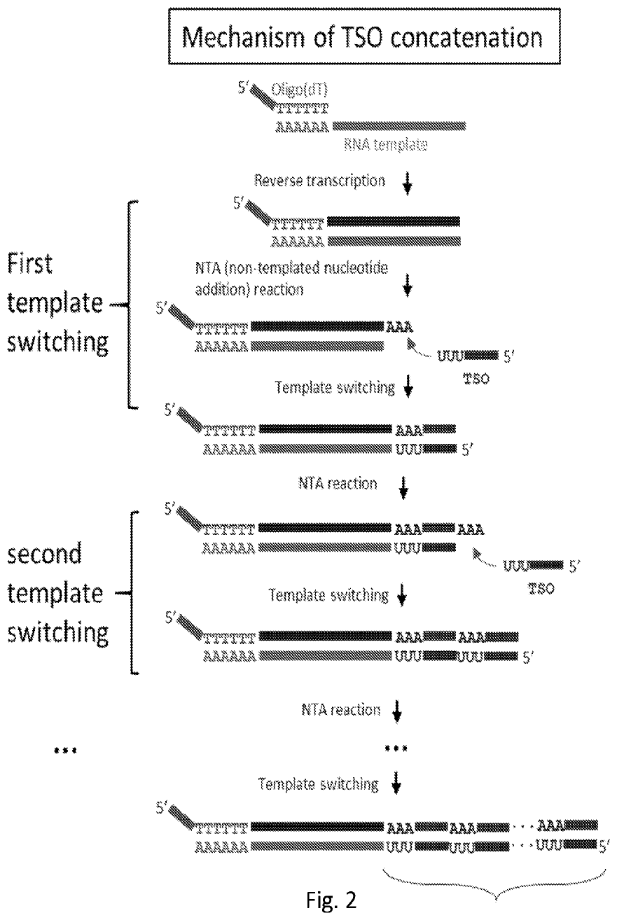

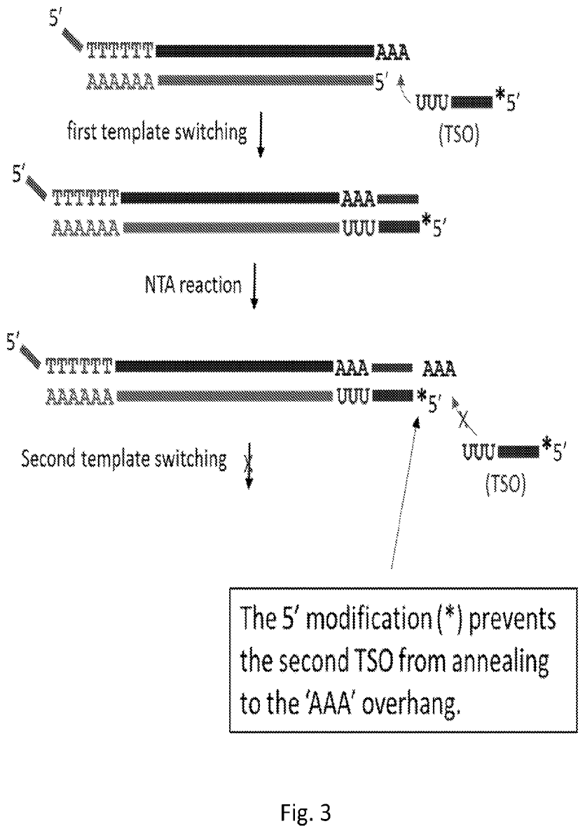

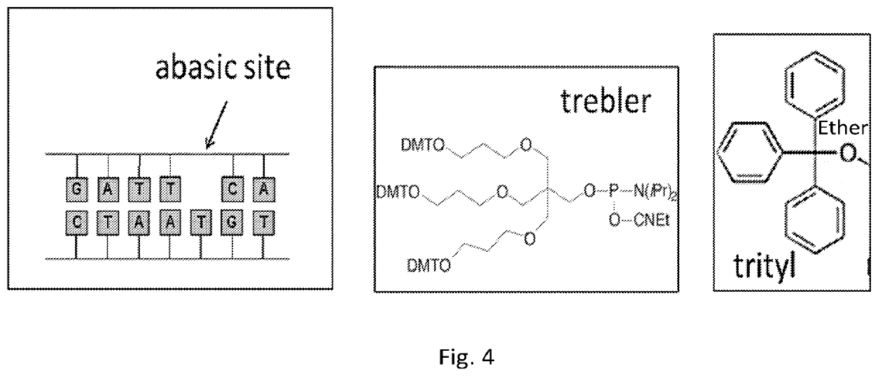

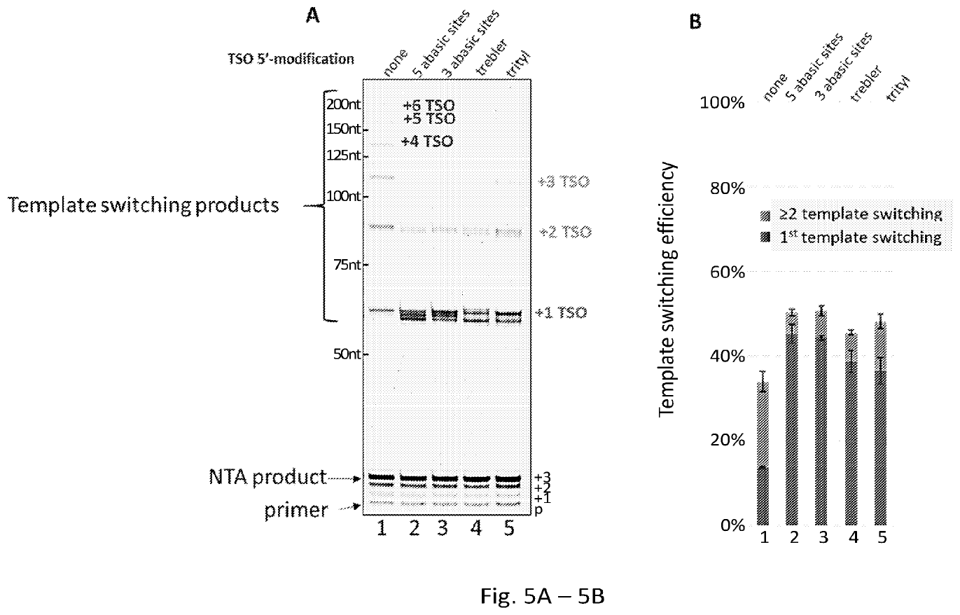

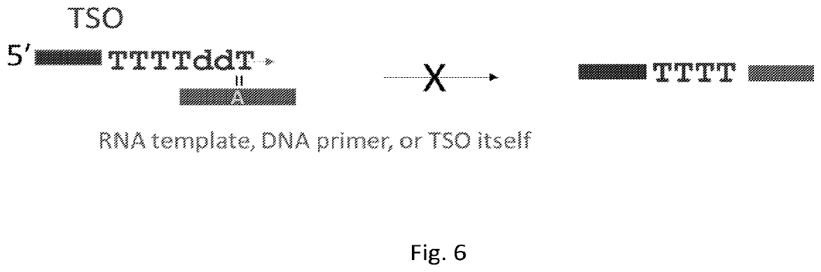

However, the TSO, as well the oligos dT primer, can also be amplified during reverse transcription and template switching, which may dominate the resulting sequencing libraries. First, during reverse transcription, the TSO can also serve as the template to be reverse transcribed by the oligo dT primer, which results in autonomous amplification of the oligos. To remove the unwanted products, the chemical composition and nucleotide sequence of the TSO and oligo dT primer were optimized to reduce the base pairing potential between the TSO and the oligo dT primer to avoid reverse transcription between them (for example, SEQ ID NO:3 and SEQ ID NO:2 respectively). Second, in addition to serving as a template, the TSO can also serve as a primer to reverse transcribe itself, and thus the hydroxyl group at the 3′ end of TSO was either removed or blocked with chemical groups that prevent extension by MarathonRT (FIG. 1 and FIG. 6). To remove the hydroxyl group at the 3′ end of TSO, dideoxythymidine was used at the 3′ end of TSO. Third, the template switching by MarathonRT is very efficient, and therefore the TSO can be tandemly concatemerization many times (>100 times) due to cycles of reverse transcriptase and terminal transferase activity (FIG. 2). To prevent TSO concatemers, one or more large chemical groups was added to the 5′ end of TSO, which effectively prevents multiple cycles of template switching by MarathonRT (FIGS. 1 and 3-5). These chemical groups include multiple (3-5) 5′AP sites (apurinic/apyrimidinic sites), 5′ trebbler, and 5′ trityl (FIG. 4 and FIG. 5). The chemical composition of this particular TSO, and its applications, is therefore novel. In summary, by performing a systemic optimization of the TSO, the template switching reaction of MarathonRT was harnessed to prepare RNA-seq libraries from ultra-low RNA input.

The group II intron encoded RT used in this system (MarathonRT or E.r. maturase) is highly processive and its activity is not affected by the sequence and structure of RNA templates during reverse transcription and template switching. Using this enzyme for reverse transcription and template switching effectively improves full-length cDNA yield and reduces the biases caused by the heterogeneity of template sequence and structure. By optimizing the reaction conditions (such as using LiCl instead of KCl and adding PEG8000), the template switching efficiency is increased from 2% to 50%), which dramatically improves cDNA yield. Efficient template switching causes concatemerization of the template switching oligo that costs a large fraction of the sequencing reads. Adding chemical modifications to or using a non-standard nucleotide at the 5′-end of the template switching oligo prevented template switching oligo concatemers, which were nearly undetectable in the sequencing reads. When using a very small amount of RNA templates, the non-specific reverse transcription of the RT primer and template switching oligo dominates the cDNA products. The nucleotide sequences of the primer and template switching oligo were optimized to minimize the base pairing potential between them. With the systematic optimization, in an RNA sequencing experiment, the optimized method provides sensitive and quantitative detection of RNA transcripts.

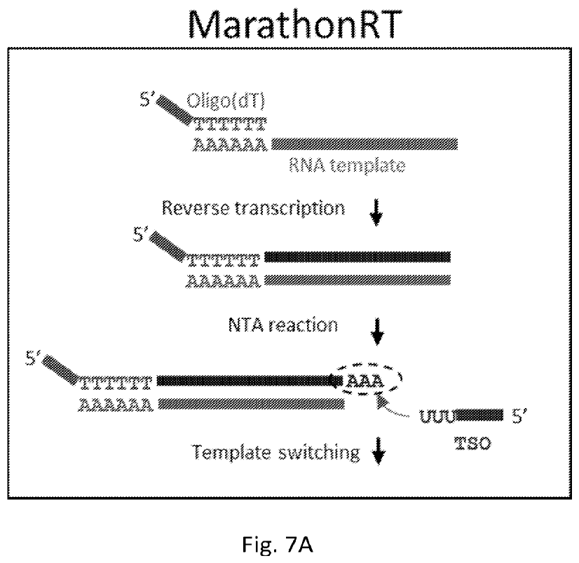

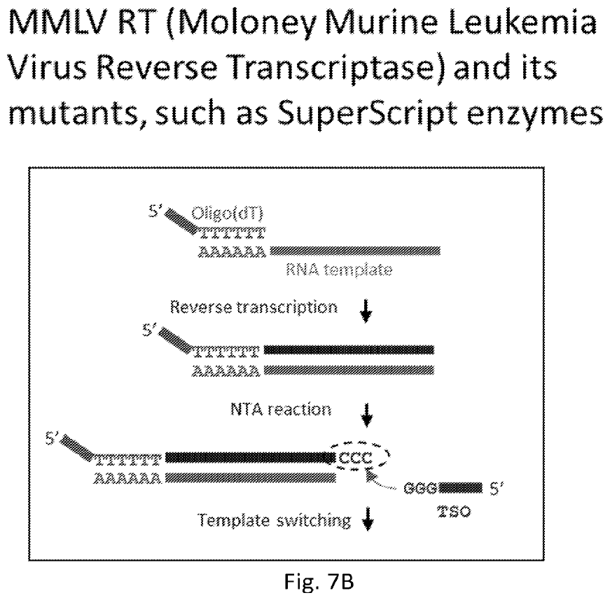

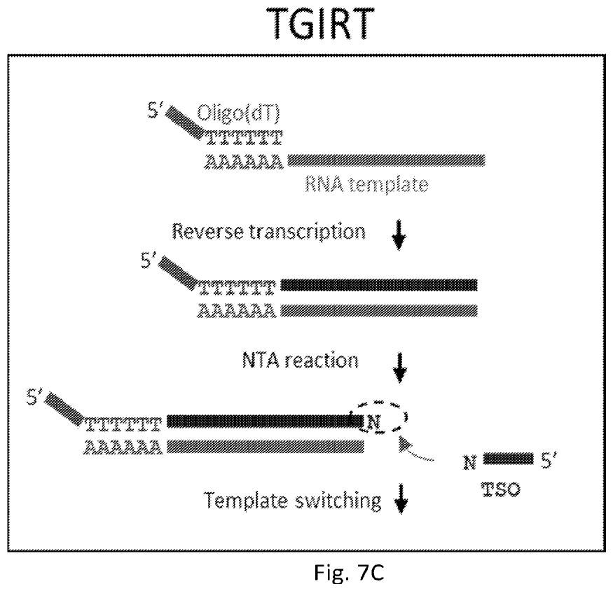

FIGS. 7A through 7C show that modifications of the TSO that can be used for performing reverse transcription with many types of reverse transcriptases. Different reverse transcriptases have a preference for different non-templated nucleotide addition (NTA). The sequence of the TSO can be selected based on the standard NTA by the reverse transcriptase. MarathonRT specifically adds a triple-adenosine overhang to the 3′-end of cDNA. It needs a TSO with three uridines (RNA) or three thymidines (DNA) at the 3′-end for efficient template switching. MMLV RTs adds a triple-cytidine overhang to the 3′-end of cDNA. It needs a TSO with three guanosines at the 3′-end for efficient template switching. TGIRT™ most efficiently adds a single nucleotide overhang (a mixture of A, G, C and T) to the 3′-end of cDNA. A TSO with any nucleotide at the 3′-end can mediate template switching.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A template switching oligonucleotide (TSO) comprising:
(i) a DNA nucleotide sequence, an RNA nucleotide sequence, or a hybrid DNA-RNA sequence; and
(ii) at least one of a 3′ end modification and a 5′ end modification.

2. The TSO of claim 1, wherein the TSO comprises a 3′ end modification.

3. The TSO of claim 1, wherein the TSO comprises a 5′ end modification.

4. The TSO of claim 1, wherein the TSO comprises both a 3′ end modification and a 5′ end modification.

5. The TSO of claim 1, wherein the 3′ end modification is a nucleotide sugar modification or a nucleobase modification.

6. The TSO of claim 1, wherein the 3′ end modification is selected from the group consisting of a modification to remove the 3′ hydroxyl group and a modification to block the 3′ hydroxyl group.

7. The TSO of claim 1, wherein the 3′ end modification is selected from the group consisting of 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1).

8. The TSO of claim 1, wherein the 5′ end modification is a nucleotide sugar modification or a nucleobase modification.

9. The TSO of claim 1, wherein the 5′ end modification comprises trityl, trebbler, a dendrimer, biotin, a fluorescent dye, ROX NHS ester, (CH2)n (n≥1) long spacer, palmitate phosphoramidite, 3-cyanovinylcarbazole phosphoramidite, cholesteryl, or psoralen.

10. The TSO of claim 1, wherein the 3′ end modification is selected from the group consisting of a modification to remove the 3′ hydroxyl group and a modification to block the 3′ hydroxyl group.

11. The TSO of claim 1, wherein the 5′ end comprises at least 2, 3, 4, 5, 6, 7, 8, 9, or 10 consecutive abasic sites.

12. The TSO of claim 1, wherein the 5′ end comprises at least 5 consecutive abasic sites.

13. The TSO of claim 1, wherein the 5′ end comprises at least 3 consecutive abasic sites.

14. The TSO of claim 1, wherein the 5′ end comprises at least one non-natural nucleotide or nucleotide analog.

15. The TSO of claim 1, wherein the TSO comprises at least one isodeoxycytosine (iso-dC), isodeoxyguanosine (iso-dG) or a combination of iso-dC and iso-dG at the 5′ end.

16. A template switching oligonucleotide (TSO) comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification.

17. The TSO of claim 16, wherein the 3′ end modification is selected from the group consisting of a modification to remove the 3′ hydroxyl group and a modification to block the 3′ hydroxyl group.

18. The TSO of claim 16, wherein the TSO comprises a 3′ end modification selected from the group consisting of 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1).

19. The TSO of claim 16, wherein the 5′ end is modified with a chemical group to block concatemerization.

20. The TSO of claim 16, wherein the 5′ end is modified with a chemical group selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

21. The TSO of claim 16, wherein the 5′ end comprises at least 3 consecutive abasic sites.

22. The TSO of claim 16, wherein the 5′ end comprises at least one non-natural nucleotide or nucleotide analog.

23. The TSO of claim 16, wherein the TSO comprises at least one isodeoxycytosine (iso-dC), isodeoxyguanosine (iso-dG) or a combination of iso-dC and iso-dG at the 5′ end.

24. The TSO of claim 16, wherein the TSO comprises at least one 3′ end modification and at least one 5′ end modification.

25. A reverse transcription (RT) primer comprising a DNA nucleotide sequence, an RNA nucleotide sequence, a modified nucleotide sequence or a hybrid DNA-RNA sequence, wherein the RT primer comprises a 5′ end modification.

26. The RT primer of claim 25, wherein the 5′ end is modified with a chemical group to block concatemerization.

27. The RT primer of claim 25, wherein the 5′ end is modified with a chemical group selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

28. The RT primer of claim 25, wherein the 5′ end comprises at least 3 consecutive abasic sites.

29. The RT primer of claim 25, wherein the 5′ end comprises at least one non-natural nucleotide or nucleotide analog.

30. The RT primer of claim 25, wherein the RT primer comprises at least one isodeoxycytosine (iso-dC), isodeoxyguanosine (iso-dG) or a combination of iso-dC and iso-dG at the 5′ end.

31. A method of generating a cDNA molecule from an RNA template, the method comprising contacting an RNA template with a TSO of claim 25, a reverse transcription (RT) primer and a reverse transcriptase.

32. The method of claim 31, wherein the RT primer comprises a DNA nucleotide sequence, an RNA nucleotide sequence, a modified nucleotide sequence or a hybrid DNA-RNA sequence, wherein the RT primer comprises a 5′ end modification.

33. The method of claim 31 wherein the reverse transcriptase is selected from the group consisting of MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, and TGIRT™ or a variant thereof.

34. The method of claim 31, wherein the method is included in an assay selected from the group consisting of group RT-PCR, qRT-PCR, capillary electrophoresis (CE) for RNA-structure mapping, transcriptome profiling, in-cell sequencing, next-generation RNA sequencing (RNA-seq), nanopore sequencing, PacBio sequencing, zero-mode waveguide sequencing, cDNA library synthesis, cDNA synthesis, or any combination thereof.

35. A reverse transcription assay for of generating a cDNA molecule from an RNA template, the method comprising contacting an RNA template with a TSO comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification, a reverse transcription (RT) primer and a reverse transcriptase.

36. The method of claim 35, wherein the RT primer is selected from the group consisting of a DNA primer, an RNA primer, a primer comprising at least one modified oligonucleotide, and an RT primer of any one of claims 10-15.

37. The assay of claim 35, wherein the reverse transcriptase is selected from the group consisting of MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, and TGIRT™ or a variant thereof.

38. The assay of claim 35, wherein the reverse transcription is performed in a buffer comprising PEG8000.

39. The assay of claim 35, wherein the reverse transcription is performed in a buffer comprising LiCl.

40. A kit for performing an assay for generating a cDNA molecule from an RNA template, the method comprising contacting an RNA template with a TSO comprising a DNA nucleotide sequence, an RNA nucleotide sequence, a modified nucleotide sequence or a hybrid DNA-RNA sequence, wherein the TSO can anneal by base-pairing to non-templated nucleotides that have been added to the 5′-end of a target nucleic acid molecule during a non-templated addition by a reverse transcriptase, and wherein the TSO further comprises at least one of a 3′ end modification and a 5′ end modification, a reverse transcription (RT) primer and a reverse transcriptase.

41. The kit of claim 40, wherein the RT primer is selected from the group consisting of a DNA primer, an RNA primer, a primer comprising at least one modified oligonucleotide, and an RT primer of any one of claims 10-15.

42. The kit of claim 40, wherein the reverse transcriptase is selected from the group consisting of MarathonRT or a variant thereof, Moloney Murine Leukemia Virus reverse transcriptase (MMLV RT) or a variant thereof, Avian Myeloblastosis Virus reverse transcriptase (AMV RT) or a variant thereof, HIV reverse transcriptase (HIV RT) or a variant thereof, Bombyx mori R2 RNA element reverse transcriptase (R2 RT) or a variant thereof, and TGIRT™ or a variant thereof.

43. The kit of claim 40, wherein the kit comprises a buffer comprising PEG8000.

44. The kit of claim 40, wherein the kit comprises a buffer comprising LiCl.

45. A method of reducing the concatemerization of a template switching oligonucleotide (TSO), the method comprising providing a reaction mixture comprising a TSO, a reverse transcription (RT) primer, and a reverse transcriptase, wherein the TSO comprises:
(i) a 3′ end modification; and/or
(ii) a 5′ end modification.

46. The method of claim 45, wherein the concatemerization is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

47. The method of claim 45, wherein the 3′ end modification is selected from the group consisting of 3′ ddT, 3′ ddU, 3′ Inverted dT, 3′ C3 spacer, 3′ amino, 3′ rU oxidized by periodate, 3′ phosphorylation, 3′ fluoro, 3′ aldehyde, 3′ carboxylate, 3′ thiol, 3′ O-methyl, 3′ azido, 3′ alkyne, 3′ alkene, 3′ (CH2)n-X (X=H, OCH3, CH3, SH, NH2, OH, etc.; n≥1), and 3′ (CH2CH2O)n (n≥1).

48. The method of claim 45, wherein the TSO comprises a 5′ end modification selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

49. The method of claim 45, wherein the 5′ end of the TSO comprises at least 3 consecutive abasic sites.

50. The method of claim 45, wherein the TSO comprises at least one isodeoxycytosine (iso-dC), isodeoxyguanosine (iso-dG) or a combination of iso-dC and iso-dG at the 5′ end.

51. The method of claim 45, wherein the TSO comprises SEQ ID NO: 3.

52. The method of claim 45, wherein the RT primer comprises SEQ ID NO: 2.

53. A method of reducing the non-specific reverse transcription from a template switching oligonucleotide (TSO), the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

54. The method of claim 49, wherein the non-specific reverse transcription is reduced by about 1%, 2%, 3%, 4%, 5%, 10%, 15%, 20%, 25%, 30%, 40%, 50%, 60%, 70%, 80%, 90%, 95%, 99%, e.g., according to a reference standard.

55. The method of claim 53, wherein TSO comprises a 5′ end modification selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

56. The method of claim 53, wherein the 5′ end of the TSO comprises at least 3 consecutive abasic sites.

57. The method of claim 53, wherein the TSO comprises at least one isodeoxycytosine (iso-dC), isodeoxyguanosine (iso-dG) or a combination of iso-dC and iso-dG at the 5′ end.

58. The method of claim 53, wherein the TSO comprises SEQ ID NO: 3.

59. The method of claim 53, wherein the RT primer comprises SEQ ID NO: 2.

60. A method of increasing yield of target polynucleotide sequences in a RNA-seq library, the method comprising providing a reaction mixture comprising a TSO, a reverse transcription (RT) primer, and a reverse transcriptase,
wherein the TSO comprises:
(i) a 3′ end modification; and/or
(ii) a 5′ end modification.

61. The method of claim 60, wherein the yield of the target polynucleotide is increased by about 1%, 2%, 3%, 4%, 5%, 10%, 15%, 20%, 25%, 30%, 40%, 50%, 60%, 70%, 80%, 90%, 95%, 99%, e.g., according to a reference standard.

62. The method of claim 60, wherein the TSO comprises a 5′ end modification selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

63. The method of claim 60, wherein the 5′ end of the TSO comprises at least 3 consecutive abasic sites.

64. The method of claim 60, wherein the TSO comprises at least one isodeoxycytosine (iso-dC), isodeoxyguanosine (iso-dG) or a combination of iso-dC and iso-dG at the 5′ end.

65. The method of claim 60, wherein the TSO comprises SEQ ID NO: 3.

66. The method of claim 60, wherein the RT primer comprises SEQ ID NO: 2.

67. A method of increasing the specificity of an RNA-seq library, the method comprising providing a reaction mixture comprising a TSO, a reverse transcription (RT) primer, and a reverse transcriptase,
wherein the TSO comprises:
(i) a 3′ end modification; and/or
(ii) a 5′ end modification;
wherein the RNA-seq library is prepared using template switching.

68. The method of claim 67, wherein the specificity of the RNA-seq library is increased by about 1%, 2%, 3%, 4%, 5%, 10%, 15%, 20%, 25%, 30%, 40%, 50%, 60%, 70%, 80%, 90%, 95%, 99%, e.g., according to a reference standard.

69. The method of claim 67, wherein the TSO comprises a 5′ end modification selected from the group consisting of trityl, trebbler, dendrimers, biotin, fluorescent dyes, ROX NHS ester, (CH2)n (n≥1) long spacer, palmitate phosphoramidite, 3-cyanovinylcarbazole phosphoramidite, cholesteryl, and psoralen.

70. The method of claim 67, wherein the 5′ end of the TSO comprises at least 3 consecutive abasic sites.

71. The method of claim 67, wherein the TSO comprises at least one isodeoxycytosine (iso-dC), isodeoxyguanosine (iso-dG) or a combination of iso-dC and iso-dG at the 5′ end.

72. The method of claim 67, wherein the TSO comprises SEQ ID NO: 3.

73. The method of claim 67, wherein the RT primer comprises SEQ ID NO: 2.
